(19) United States
(12) Patent Application Publication
(10) Pub. No.: US 2025/0114001 A1
(43) Pub. Date: Apr. 10, 2025

(54) COMMUNICATION TECHNOLOGY FOR ANALYTE MONITORING SYSTEM

(71) Applicant: Dexcom, Inc., San Diego, CA (US)

(72) Inventors: Gary Thomas NEEL, Dania Beach, FL (US); Amit VERMA, Dania Beach, FL (US); Javaid MASOUD, San Diego, CA (US)

(21) Appl. No.: 18/887,956

(22) Filed: Sep. 17, 2024

Related U.S. Application Data
(60) Provisional application No. 63/589,270, filed on Oct. 10, 2023.

(51) Int. Cl. A61B 5/00; A61B 5/145; H04B 17/318

(52) U.S. Cl. CPC A61B 5/0004; A61B 5/145; H04B 17/318

(57) ABSTRACT

Aspects of the present disclosure provide techniques for improving a communication range of an analyte sensor system. The analyte sensor system may include a analyte sensor configured to generate analyte data associated with analyte levels of a user of the analyte sensor system, an antenna system comprising a plurality of antennas, a transceiver circuit configured to transmit the analyte data to a communications device via one or more antennas of the plurality of antennas of the antenna system, a switching device configured to selectively couple the one or more antennas to the transceiver circuit, and a circuit board configured to operatively connect the transcutaneous analyte sensor with the transceiver circuit.

### CROSS-REFERENCE TO RELATED APPLICATIONS

This application claims priority to and benefit of U.S. Provisional Patent Application No. 63/589,270, filed Oct. 10, 2023, which is hereby assigned to the assignee hereof and hereby expressly incorporated by reference herein in its entirety as if fully set forth below and for all applicable purposes.

### TECHNICAL FIELD

The present disclosure relates generally to an electronic device, such as an analyte sensor system for monitoring analyte values of a user.

### BACKGROUND

Diabetes is a metabolic condition relating to the production or use of insulin by the body. Insulin is a hormone that allows the body to use glucose for energy, or store glucose as fat. When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher-than-normal blood sugar level is called “hyperglycemia.” Chronic hyperglycemia can lead to several of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce adequate insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

### SUMMARY

Aspects of the present disclosure provide techniques for improving a communication range of an analyte sensor system. The analyte sensor system may include an analyte sensor configured to generate analyte data associated with analyte levels of a user of the analyte sensor system, an antenna system comprising a plurality of antennas, a transceiver circuit configured to transmit the analyte data to a communications device via one or more antennas of the plurality of antennas, a switching device configured to selectively couple the one or more antennas to the transceiver circuit, and a circuit board configured to operatively connect the analyte sensor with the transceiver circuit.

Additional aspects relate to an antenna system for communicating analyte data. The antenna system may include a plurality of antennas and a switching device configured to selectively couple one or more antennas of the plurality of antennas to a transceiver circuit of an analyte sensor system. In some embodiments, when selectively coupled to the transceiver circuit of the analyte sensor system, the one or more antennas are configured to: receive, from an analyte sensor of the analyte sensor system via the transceiver circuit and a circuit board, analyte data associated with analyte levels of a user of the analyte sensor system; and transmit the analyte data to a communications device for display to the user.

Additional aspects relate to an analyte monitoring system. The analyte monitoring system may include a communications device and an analyte sensor system. In some embodiments, the analyte sensor system may include an analyte sensor configured to generate analyte data associated with analyte levels of a user of the analyte sensor system. a first antenna system comprising plurality of antennas, a transceiver circuit configured to transmit the analyte data to a communications device via one or more antennas of the plurality of antennas of the antenna system, a switching device configured to selectively couple the one or more antennas to the transceiver circuit, and a circuit board configured to operatively connect the analyte sensor with the transceiver circuit. In some embodiments, the communications device comprises a second antenna system configured to receive the analyte data from the first antenna system of the analyte sensor system. In some embodiments, the communications device is configured to display the analyte data received from the first antenna system of the analyte sensor system to the user.

Additional aspects relate to a method for communication by an analyte sensor system. The method includes generating analyte data associated with analyte levels of a user of the analyte sensor system, selecting, using a switching device of the analyte sensor system, a first antenna of a plurality of antennas of an antenna system of the analyte sensor system for transmission of the analyte data, transmitting the analyte data to a communications device using the first antenna, selecting, using the switching device, at least a second antenna of the plurality of antennas of the antenna system, and transmitting the analyte data to the communications device using at least the second antenna.

Additional aspects relate to a method for communication between a communications device and an analyte sensor system in an analyte monitoring system. The method includes receiving, by the communications device, analyte data from the analyte sensor system in a first position, failing to receive, by the communications device, the analyte data from the analyte sensor system in a second position different from the first position, switching, by the analyte sensor system, an antenna for transmission of the analyte data, and receiving, by the communications device, the analyte data from the analyte sensor system in the second position based on switching the antenna.

### BRIEF DESCRIPTION OF THE DRAWINGS

Further aspects of the present disclosure will be more readily appreciated upon review of the detailed description of the various disclosed embodiments, described below, when taken in conjunction with the accompanying figures.

FIG. 1 illustrates aspects of an example system that may be used in connection with some embodiments.

FIG. 2 illustrates aspects of an example system that may be used in connection with some embodiments.

FIG. 3A is an example analyte sensor system, in accordance with some embodiments.

FIG. 3B is an example analyte sensor system, in accordance with some embodiments.

FIG. 4 illustrates aspects of an example analyte sensor system, in accordance with some embodiments.

FIG. 5 illustrates aspects of an example analyte sensor system, in accordance with some embodiments.

FIG. 6 illustrates an example radiation pattern of one or more antennas of an analyte sensor system.

FIG. 7 illustrates an example analyte sensor system incorporating a plurality of antennas for spatial diversity.

FIG. 8 illustrates an example embodiment in which angle-of-arrival information is used to select an antenna of the analyte sensor system.

FIGS. 9, 10A, and 10B illustrate example embodiments related to operating antennas of the analyte sensor system in different manners.

FIG. 11 depicts a method for wireless communication by an analyte sensor system, according to some embodiments disclosed herein.

FIG. 12 depicts a method for communication between a communications device and an analyte sensor system in an analyte monitoring system, according to some embodiments disclosed herein.

FIG. 13 depicts aspects of an example health monitoring device, according to some embodiments disclosed herein.

FIG. 14 depicts aspects of an example health monitoring device, according to some embodiments disclosed herein.

The figures, described in greater detail in the description and examples below, are provided for purposes of illustration only, and merely depict typical or example embodiments of the disclosure. The figures are not intended to be exhaustive or to limit the disclosure to the precise form disclosed. It should also be understood that the disclosure may be practiced with modification or alteration, and that the disclosure may be limited only by the claims and the equivalents thereof.

### DETAILED DESCRIPTION

Aspects of the present disclosure provide systems, methods, and devices for improving a communication range of an analyte sensor system. For example, analyte sensor systems may be worn by users and are configured to continuously monitor analyte levels of the user. These data indicating these analyte levels may then be transmitted from the analyte sensor system to a display device (e.g., smart phone) using one or more antennas, allowing the user to conveniently track their analyte levels. Certain existing analyte sensor systems are bulky and tend to be uncomfortable to wear. As a result, there is a constant competitive drive to miniaturize analyte sensor systems, for example, to provide better comfort, discreet usage, and/or ease of use to the user.

However, this miniaturization may have negative effects on communication or transmission ranges of the analyte sensor systems, which may result in the display device not receiving the analyte levels of the user. In some cases, these negative effects may be the result of the one or more antennas of the analyte sensor system being positioned closer to the body of the user. For example, when one or more antennas are positioned closer to the body of the user, a significant portion of radio frequency (RF) power radiated by the one or more antennas during analyte data transmission may be absorbed by the body of the user, which significantly reduces the transmission or communication range of the analyte sensor system.

Accordingly, aspects of the present disclosure provide techniques for improving a communication or transmission range of an analyte sensor system. In some embodiments, these techniques may involve using a spatially diverse antenna architecture to enhance wireless communication performance of the analyte sensor system. For example, the analyte sensor system may include a plurality of antennas, each of which may be arranged in different positions within the analyte sensor system to facilitate spatial diversity in radiation patterns of the analyte sensor system. For example, the plurality of antennas may include a first antenna, which may provide a radiation pattern oriented in a first spatial direction with a high directivity to facilitate an increased transmission range within the first spatial direction. Additionally, the plurality of antennas may include a second antenna, which may provide a radiation pattern oriented in a second spatial direction (different from the first spatial direction) with a high directivity to facilitate an increased transmission range in the second spatial direction. In some embodiments, the analyte sensor system may also include a third antenna formed through combination of the first antenna and the second antenna, which may provide a pseudo or near omni-directional radiation pattern.

The details of some example embodiments of the systems, methods, and devices of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

### System Overview and Example Configurations

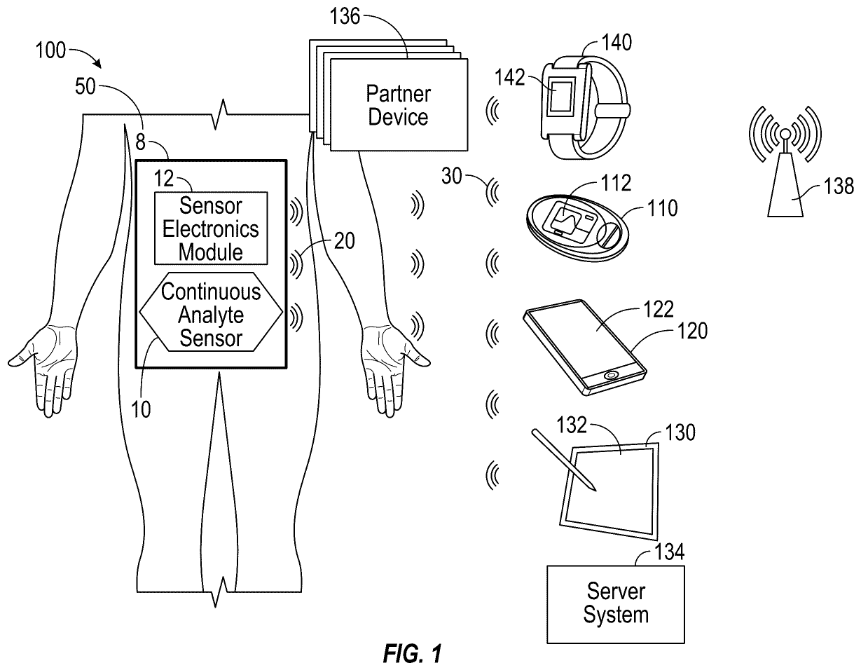

FIG. 1 depicts a system 100 that may be used in connection with embodiments of the present disclosure that involve gathering, monitoring, and/or providing information regarding analyte values present in a user's body, including for example the user's blood glucose values, other analytes, multiple multiplexed or simultaneous measured analytes, or the like. System 100 depicts aspects of analyte sensor system 8 that may be communicatively coupled to one or more communications devices, such as display devices 110, 120, 130, and 140, partner devices 136, and/or server system 134.

Analyte sensor system 8 in the illustrated embodiment includes analyte sensor electronics module 12 and analyte sensor 10 associated with analyte sensor electronics module 12. Analyte sensor electronics module 12 may be electrically and mechanically coupled to analyte sensor 10 before analyte sensor 10 is implanted in a user or host. Accordingly, analyte sensor 10 may not require a user to couple analyte sensor electronics module 12 to analyte sensor 10. For example, analyte sensor electronics module 12 may be physically/mechanically and electrically coupled to analyte sensor 10 during manufacturing, and this physical/mechanical and electrical connection may be maintained during shipping, storage, insertion, use, and removal of analyte sensor system 8.

As such, the electro-mechanically connected components (e.g., analyte sensor 10 and analyte sensor electronics module 12) of analyte sensor system 8 may be referred to as a “pre-connected” system. Analyte sensor electronics module 12 may be in wireless communication (e.g., directly or indirectly) with one or more of display devices 110, 120, 130, and 140. In addition, or alternatively to display devices 110, 120, 130, and 140, analyte sensor electronics module 12 may be in wireless communication (e.g., directly or indirectly) with partner devices 136 and/or server system 134. Likewise, in some examples, display devices 110-140 may additionally or alternatively be in wireless communication (e.g., directly or indirectly) with partner devices 136 and/or server system 134. Various couplings shown in FIG. 1 can be facilitated with wireless access point (WAP) 138, as also mentioned below.

In certain embodiments, analyte sensor electronics module 12 includes electronic circuitry associated with measuring and processing analyte sensor data or information, including prospective algorithms associated with processing and/or calibration of the analyte sensor data/information. Analyte sensor electronics module 12 can be physically/mechanically connected to analyte sensor 10 and can be integral with (non-releasably attached to) or releasably attachable to analyte sensor 10. Analyte sensor electronics module 12 may also be electrically coupled to analyte sensor 10, such that the components may be electromechanically coupled to one another. Analyte sensor electronics module 12 may include hardware, firmware, and/or software that enables measurement and/or estimation of levels of the analyte in a host/user via analyte sensor 10 (e.g., which may be/include a glucose sensor). For example, analyte sensor electronics module 12 can include one or more of a potentiostat, a power source for providing power to analyte sensor 10, other components useful for signal processing and data storage, and a telemetry module for transmitting data from the sensor electronics module to one or more display devices. Electronics can be affixed to a printed circuit board (PCB) within analyte sensor system 8, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 12 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

With further reference to FIG. 1, display devices 110, 120, 130, and/or 140 can be configured for displaying (and/or alarming) displayable sensor information that may be transmitted by analyte sensor electronics module 12 (e.g., in a customized data package that is transmitted to the display devices based on their respective preferences). Each of display devices 110, 120, 130, or 140 can (respectively) include a display such as touchscreen display 112, 122, 132, or 142 for displaying sensor information and/or analyte data to a user and/or receiving inputs from the user. For example, a graphical user interface (GUI) may be presented to the user for such purposes. In embodiments, the display devices may include other types of user interfaces such as voice user interface instead of or in addition to a touchscreen display for communicating sensor information to the user of the display device and/or receiving user inputs. In embodiments, one, some, or all of display devices 110, 120, 130, 140 may be configured to display or otherwise communicate the sensor information as it is communicated from analyte sensor electronics module 12 (e.g., in a data package that is transmitted to respective display devices), without any additional prospective processing required for calibration and/or real-time display of the sensor data.

The plurality of display devices 110, 120, 130, 140 depicted in FIG. 1 may include a custom display device, for example, analyte display device 110, specially designed for displaying certain types of displayable sensor information associated with analyte data received from analyte sensor electronics module 12 (e.g., a numerical value and/or an arrow, in embodiments). In embodiments, one of the plurality of display devices 110, 120, 130, 140 includes a smartphone, such as a mobile phone, based on an Android, iOS, or other operating system, and configured to display a graphical representation of the continuous sensor data (e.g., including current and/or historic data).

As further illustrated in FIG. 1 and mentioned above, system 100 may also include WAP 138 that may be used to couple one or more of analyte sensor system 8, the plurality display devices 110, 120, 130, 140 etc., server system 134, and partner devices 136 to one another. For example, WAP 138 may provide WiFi and/or cellular or other wireless connectivity within system 100. Near Field Communication (NFC) may also be used among devices of system 100 for exchanging data, as well as for performing specialized functions, e.g., waking up or powering a device or causing the device (e.g., analyte sensor electronics module 12 and/or a transmitter) to exit a lower power mode or otherwise change states and/or enter an operational mode. Server system 134 may be used to collect analyte data from analyte sensor system 8 and/or the plurality of display devices, for example, to perform analytics thereon, generate universal or individualized models for analyte levels and profiles, provide services or feedback, including from individuals or systems remotely monitoring the analyte data, and so on.

Partner device(s) 136, by way of overview and example, can usually communicate (e.g., wirelessly) with analyte sensor system 8, including for authentication of partner device(s) 136 and/or analyte sensor system 8, as well as for the exchange of analyte data, medicament data, other data, and/or control signaling or the like. Partner devices 136 may include a passive device in example embodiments of the disclosure. One example of partner device 136 may be an insulin pump for administering insulin to a user in response and/or according to an analyte level of the user as measured/approximated using analyte sensor system 8. For a variety of reasons, it may be desirable for such an insulin pump to receive and track glucose values transmitted from analyte sensor system 8 (with reference to FIG. 1 for example). One example reason for this is to provide the insulin pump a capability to suspend/activate/control insulin administration to the user based on the user's glucose value being below/above a threshold value.

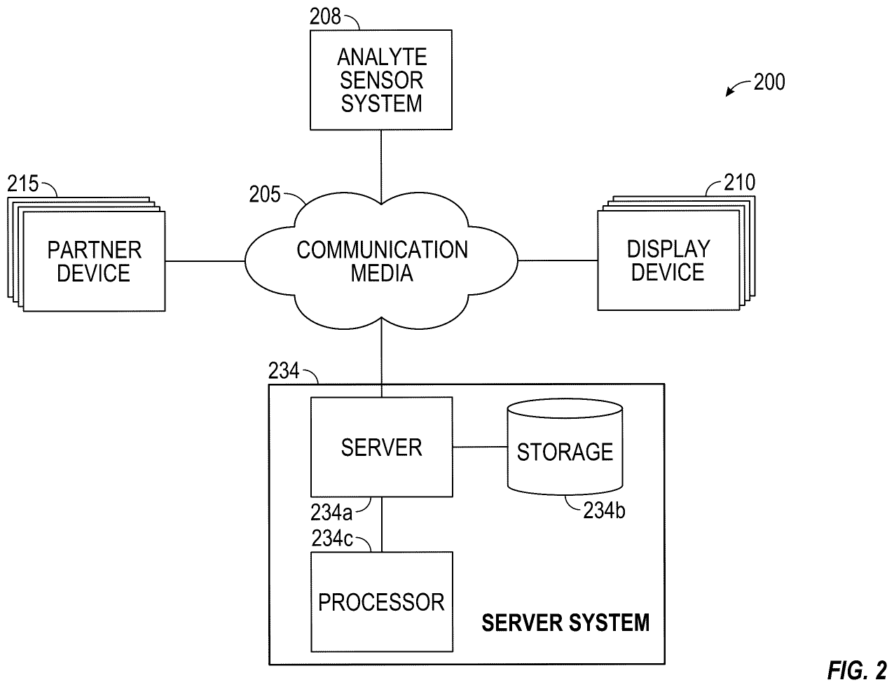

Referring now to FIG. 2, system 200 is depicted. System 200 may be used in connection with implementing embodiments of the disclosed systems, methods, apparatuses, and/or devices, including, for example, aspects described above in connection with FIG. 1. By way of example, various below-described components of FIG. 2 may be used to provide wireless communication of analyte (e.g., glucose) data, for example among/between analyte sensor system 208, display devices 210, partner devices 215, and/or one or more server systems 234, and so on. In some cases, analyte sensor system 208 illustrated in FIG. 2 may be an example of the analyte sensor system 8 illustrated in FIG. 1. Additionally, in some cases, the display devices 210 illustrated in FIG. 2 may be examples of the display devices 110, 120, 130, and 140 illustrated in FIG. 1. Additionally, in some cases, partner devices 215 illustrated in FIG. 2 may be examples of the partner device 136 illustrated in FIG. 1.

As shown in FIG. 2, system 200 may include analyte sensor system 208, one or more display devices 210, and/or one or more partner devices 215. Additionally, in the illustrated embodiment, system 200 includes server system 234, which can in turn include server 234a coupled to processor 234c and storage 234b. Analyte sensor system 208 may be coupled to display devices 210, partner devices 215, and/or server system 234 via communication media 205. Some details of the processing, gathering, and exchanging of data, and/or executing actions (e.g., providing medicaments or related instructions) by analyte sensor system 208, partner devices 215, and/or display device 210, etc., are provided below. Herein, display devices 210, partner devices 215, and server system 234 may be referred to as communications devices and may be configured to communicate with analyte sensor system 208 using a variety of communications technologies and/or protocols.

Analyte sensor system 208, display devices 210, and/or partner devices 215 may exchange messaging (e.g., control signaling) via communication media 205, and communication media 205 may also be used to deliver analyte data to display devices 210, partner devices 215, and/or server system 234. As alluded to above, display devices 210 may include a variety of electronic computing devices, such as a smartphone, tablet, laptop, wearable device, etc. Display devices 210 may also include analyte display device 110 that may be customized for the display and conveyance of analyte data and related notifications etc. Partner devices 215 may include medical devices, such as an insulin pump or pen, connectable devices, such as a smart fridge or mirror, key fob, and other devices.

In embodiments, communication media 205 may be implemented using one or more wireless communication protocols, such as for example Bluetooth, Bluetooth Low Energy (BLE), ZigBee, WiFi, IEEE 802.11 protocols, Infrared (IR), Radio Frequency (RF), 2G, 3G, 4G, 5G, etc., and/or wired protocols and media. It will also be appreciated upon studying the present disclosure that communication media can be implemented as one or more communication links, including in some cases, separate links, between the components of system 200, whether or not such links are explicitly shown in FIG. 2 or referred to in connection therewith. By way of illustration, analyte sensor system 208 may be coupled to display device 210 via a first link of communication media 205 using BLE, while analyte sensor system 208 may be coupled to server system 234 by a second link of communication media 205 using a WiFi communication protocol. In embodiments, a BLE signal may be temporarily attenuated to minimize data interceptions. For example, attenuation of a BLE signal through hardware or firmware design may occur temporarily during moments of data exchange (e.g., pairing).

In embodiments, the elements of system 200 may be used to perform operations of various processes described herein and/or may be used to execute various operations and/or features described herein with regard to one or more disclosed systems and/or methods. Upon studying the present disclosure, one of skill in the art will appreciate that system 200 may include single or multiple analyte sensor systems 208, communication media 205, and/or server systems 234.

As mentioned, communication media 205 may be used to connect or communicatively couple analyte sensor system 208, display devices 210, partner devices 215, and/or server system 234 to one another or to a network. Communication media 205 may be implemented in a variety of forms. For example, communication media 205 may include one or more of an Internet connection, such as a local area network (LAN), a person area network (PAN), a wide area network (WAN), a fiber optic network, internet over power lines, a hard-wired connection (e.g., a bus), DSL, and the like, or any other kind of network connection or communicative coupling. Communication media 205 may be implemented using any combination of routers, cables, modems, switches, fiber optics, wires, radio (e.g., microwave/RF, AM, FM links etc.), and the like. Upon reading the present disclosure, one of skill in the art will recognize other ways to implement communication media 205 for communications purposes and will also recognize that communication media 205 may be used to implement features of the present disclosure using as of yet undeveloped communications protocols that may be deployed in the future.

Further referencing FIG. 2, server 234a may receive, collect, and/or monitor information, including analyte data, medicament data, and related information, from analyte sensor system 208, partner devices 215 and/or display devices 210, such as input responsive to the analyte data or medicament data, or input received in connection with an analyte monitoring application running on analyte sensor system 208 or display device 210, or a medicament delivery application running on display device 210 or partner device 215. As such, server 234a may receive, collect, and/or monitor information from partner devices 215, such as information related to the provision of medicaments to a user and/or information regarding the operation of one or more partner devices 215. Server 234a may also receive, collect, and/or monitor information regarding a user of analyte sensor system 208, display devices 210, and/or partner devices 215.

In embodiments, server 234a may be adapted to receive such information via communication media 205. This information may be stored in storage 234b and may be processed by processor 234c. For example, processor 234c may include an analytics engine capable of performing analytics on information that server 234a has collected, received, etc. via communication media 205. The aforementioned information may then be processed at server 234a such that services may be provided to analyte sensor system 208, display devices 210, partner devices 215, and/or a user(s) thereof. For example, such services may include diabetes management feedback for the user.

With the above description of aspects of the presently disclosed systems and methods for wireless communication of analyte data, examples of some specific features of the present disclosure will now be provided. It will be appreciated by one of skill in the art upon studying the present disclosure that these features may be implemented using aspects and/or combinations of aspects of the example configurations described above, whether or not explicit reference is made to the same.

### Analyte Data

Referring back to FIG. 1, as mentioned above, in embodiments, analyte sensor system 8 is provided for measurement of an analyte in a host or user. By way of an overview and an example, analyte sensor system 8 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices (e.g., display devices 110, 120, 130, 140, partner devices 136, and/or server system 134).

Analyte sensor system 8 may include: analyte sensor 10 configured to measure a concentration or level of the analyte in the host, and analyte sensor electronics module 12 that is typically physically connected to analyte sensor 10 before analyte sensor 10 is implanted in a user. In some cases, the analyte sensor 10 may be a single-analyte sensor or a multi-analyte sensor capable of measuring one or more types of analytes, such as glucose, lactate, potassium, and/or the like. In embodiments, analyte sensor electronics module 12 includes electronics configured to process a data stream associated with an analyte concentration measured by analyte sensor 10, in order to generate sensor information that includes raw sensor data, transformed sensor data, and/or any other sensor data, for example. Analyte sensor electronics module 12 may further be configured to generate analyte sensor information that is customized for respective display devices 110, 120, 130, 140, partner devices 136, and/or server system 134. Analyte sensor electronics module 12 may further be configured such that different devices may receive different sensor information and may further be configured to wirelessly transmit sensor information to such display devices 110, 120, 130, 140, partner devices 136, and/or server system 134.

The term “analyte” as used herein is a broad term and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensor heads, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferring; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; Cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

### Analyte Sensor System

As described to above with reference to FIG. 1, in some embodiments, analyte sensor 10 includes a continuous glucose sensor, for example, a subcutaneous, transdermal (e.g., transcutaneous), or intravascular device. For example, in some embodiments, the continuous glucose sensor may be configured to continuously measure and analyze glucose measurements in the interstitial fluid. Analyte sensor 10 can use any method of analyte measurement, including for example glucose-measurement, including enzymatic, chemical, physical, electrochemical, spectrophotometric, polarimetric, calorimetric, iontophoretic, radiometric, immunochemical, and the like.

In embodiments where analyte sensor 10 is a glucose sensor, analyte sensor 10 can use any method, including invasive, minimally invasive, and non-invasive sensing techniques (e.g., fluorescence monitoring), or the like, to provide a data stream indicative of the concentration of glucose in a host. The data stream may be a raw data signal, which may be converted into a calibrated and/or filtered data stream that can be used to provide a useful value of glucose to a user, such as a patient or a caretaker (e.g., a parent, a relative, a guardian, a teacher, a doctor, a nurse, or any other individual that has an interest in the wellbeing of the host).

A glucose sensor can be any device capable of measuring the concentration of glucose. According to one example embodiment described below, an implantable glucose sensor may be used. However, it should be understood that the devices and methods described herein can be applied to any device capable of detecting a concentration of an analyte, glucose for example, and providing an output signal that represents the concentration of the analyte, again glucose for example (e.g., as a form of analyte data).

In embodiments, analyte sensor 10 is an implantable glucose sensor, such as described with reference to U.S. Pat. No. 6,001,067 and U.S. Patent Publication No. US-2005-0027463-A1. In embodiments, analyte sensor 10 is a transcutaneous glucose sensor, such as described with reference to U.S. Patent Publication No. US-2006-0020187-A1. In embodiments, analyte sensor 10 is configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-A1 filed on Feb. 14, 2007. In embodiments, the continuous glucose sensor includes a transcutaneous sensor such as described in U.S. Pat. No. 6,565,509 to Say et al., for example. In embodiments, analyte sensor 10 is a continuous glucose sensor that includes a subcutaneous sensor such as described with reference to U.S. Pat. No. 6,579,690 to Bonnecaze et al. or U.S. Pat. No. 6,484,046 to Say et al., for example. In embodiments, the continuous glucose sensor includes a refillable subcutaneous sensor such as described with reference to U.S. Pat. No. 6,512,939 to Colvin et al., for example. The continuous glucose sensor may include an intravascular sensor such as described with reference to U.S. Pat. No. 6,477,395 to Schulman et al., for example. The continuous glucose sensor may include an intravascular sensor such as described with reference to U.S. Pat. No. 6,424,847 to Mastrototaro et al., for example.

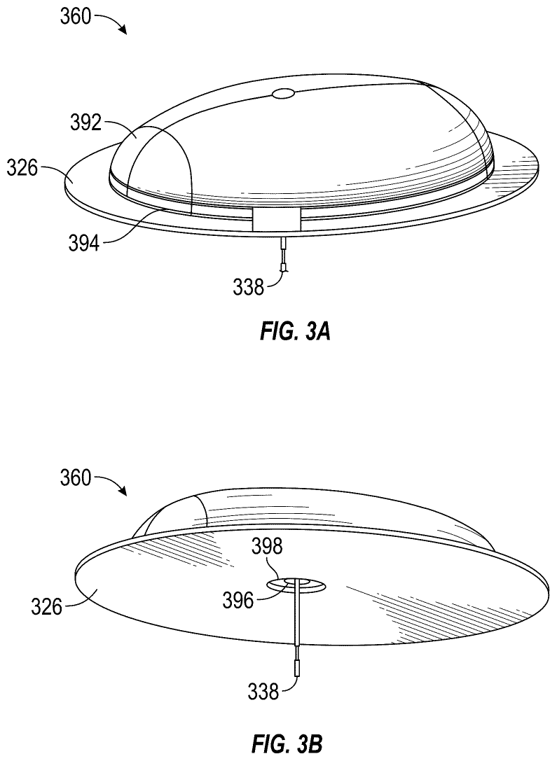

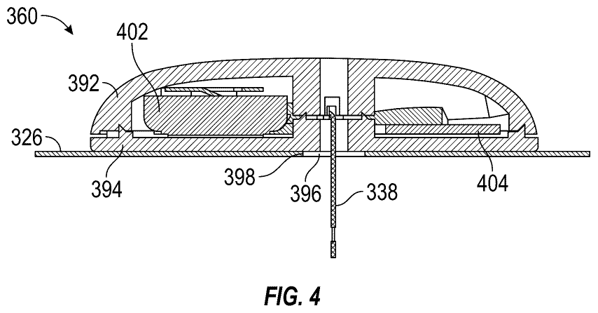

FIG. 3A illustrates a perspective view of an on-skin sensor assembly 360 that may be used in connection with the analyte sensor system 8 of FIG. 1 and/or the analyte sensor system 208 of FIG. 2. For example, on-skin sensor assembly 360 may be or include analyte sensor system 8 and/or analyte sensor system 208. On-skin sensor assembly 360 may include an outer housing with a first, top portion 392 and a second, bottom portion 394. In embodiments, the outer housing may include a clamshell design. On-skin sensor assembly 360 may include, for example, similar components as analyte sensor electronics module 12 described above in connection with FIG. 1, for example, a potentiostat, a power source for providing power to analyte sensor 10, signal processing components, data storage components, and a communication module (e.g., a telemetry module) for one-way or two-way data communication, a printed circuit board (PCB), an integrated circuit (IC), an Application-Specific Integrated Circuit (ASIC), a microcontroller, and/or a processor.

As shown in FIG. 3A, the outer housing may feature a generally oblong shape. The outer housing may further include aperture 396 disposed substantially through a center portion of outer housing and adapted for sensor 338 and needle insertion through a bottom of on-skin sensor assembly 360. In embodiments, aperture 396 may be a channel or elongated slot. On-skin sensor assembly 360 may further include an adhesive patch 326 configured to secure on-skin sensor assembly 360 to skin of the host. In embodiments, adhesive patch 326 may include an adhesive suitable for skin adhesion, for example a pressure sensitive adhesive (e.g., acrylic, rubber-based, or other suitable type) bonded to a carrier substrate (e.g., spun lace polyester, polyurethane film, or other suitable type) for skin attachment, though any suitable type of adhesive is also contemplated. As shown, adhesive patch 326 may feature an aperture 398 aligned with aperture 396 such that sensor 338 may pass through a bottom of on-skin sensor assembly 360 and through adhesive patch 326.

FIG. 3B illustrates a bottom perspective view of on-skin sensor assembly 360 of FIG. 3A. FIG. 3B further illustrates aperture 396 disposed substantially in a center portion of a bottom of on-skin sensor assembly 360, and aperture 398, both adapted for sensor 338 and needle insertion.

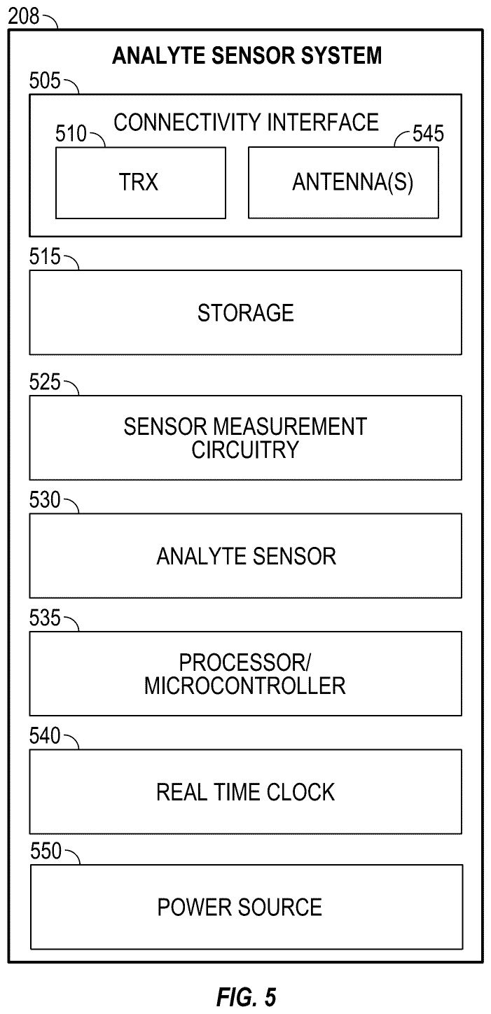

FIG. 4 illustrates a cross-sectional view of on-skin sensor assembly 360 of FIGS. 3A and 3B. FIG. 4 illustrates first, top portion 392 and second, bottom portion 394 of the outer housing, adhesive patch 326, aperture 396 in the center portion of on-skin sensor assembly 360, aperture 398 in the center portion of adhesive patch 326, and sensor 338 passing through aperture 396. The electronics unit, previously described in connection with FIG. 3A, may further include circuit board 404 and battery 402 configured to provide power to at least circuit board 404.

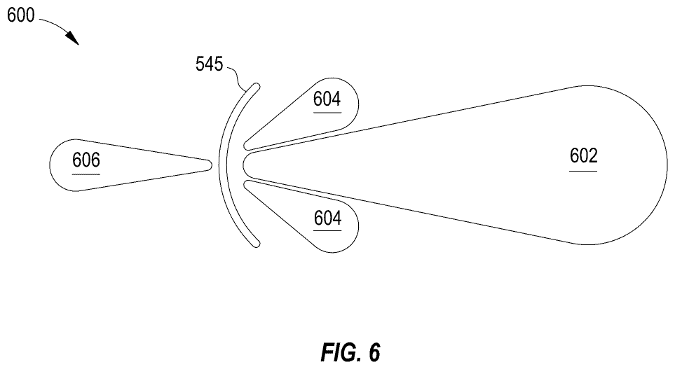

Turning now to FIG. 5, a more detailed functional block diagram of analyte sensor system 208 (discussed above, for example, in connection with FIGS. 1 and 2) is provided. As noted above, the analyte sensor system 208 may be an example of the analyte sensor system 8 illustrated in FIG. 1. As shown in FIG. 5, analyte sensor system 208 may include an analyte sensor 530 (e.g., which may be an example of the analyte sensor 10 illustrated in FIG. 1) coupled to sensor measurement circuitry 525 for receiving, processing, and managing analyte data. Sensor measurement circuitry 525 may be coupled to processor/microcontroller 535. In some embodiments, processor/microcontroller 535 may include one or more processors and may be part of analyte sensor electronics module 12 in FIG. 1. In some embodiments, processor/microcontroller 535 may perform part or all of the functions of sensor measurement circuitry 525 for obtaining and processing analyte data (e.g., sensor measurement values) from the analyte sensor 530. In some embodiments, the processed analyte data may be stored in storage 515, including one or more memories.

Processor/microcontroller 535 may be further coupled to a radio unit or transceiver 510 (e.g., which may be part of analyte sensor electronics module 12 in FIG. 1) for sending sensor and other data and receiving requests and commands and other signaling from an external device, such as display device 310 (referencing FIG. 2 by way of example). In some cases, the transceiver 510 may include logic or circuitry for communicating using different communication protocols, such as Bluetooth, Bluetooth Low Energy (BLE), near-field communication (NFC), and other wireless communication protocols. In some embodiments, the transceiver 510 may be coupled to an antenna system 545 associated with the connectivity interface 505, allowing the analyte sensor system 208 to wirelessly transmit and receive data. For example, the transceiver 510 may be configured to output data for wireless transmission to a communications device (e.g., display devices 210, the partner devices 215, or server system 234 illustrated and described with respect to FIG. 2) via at least one of the antenna system 545 or may be configured to obtain data that is wirelessly received via at least one of the antenna system 545. In some cases, one or more antennas of the antenna system 545 may be tuned to a particular frequency depending on a communication protocol used for communicating data. For example, in some embodiments, the antenna system 545 may include one or more antennas tuned for communicating data via a BLE protocol (e.g., tuned to 2.4 gigahertz). In some embodiments, the antenna system 545 may include one or more antennas tuned for communicating data via an NFC protocol (e.g., tuned to 13.56 megahertz).

Analyte sensor system 208, in example implementations, gathers analyte data using the analyte sensor 530 and transmits the same or a derivative thereof to a communications device, such as display device 210, partner device 215, and/or server system 234 using the transceiver 510 and antenna system 545. Data points regarding analyte values may be gathered and transmitted over the life of the analyte sensor 530. New measurements and/or related information may be transmitted often enough for a remote device/individual to adequately monitor analyte (e.g., glucose) levels.

It is to be appreciated that some details of the processing, gathering, and exchanging data by analyte sensor system 208, partner devices 215, and/or display device 210 etc. are provided elsewhere herein. It will be appreciated upon studying the present disclosure that analyte sensor system 208 may contain several like components that are described with respect to FIG. 1 or 2, at least for some embodiments herein. The details and uses of such like components may therefore be understood vis-a-vis analyte sensor system 208 even if not expressly described here with reference to FIG. 5.

### Aspects Related to a Spatially Diverse Antenna System for an Analyte Sensor System

Patients with diabetes may benefit from real-time diabetes management guidance that is determined based on a physiological state of the patient. In certain cases, the physiological state of the patient is determined using monitoring systems, such as an analyte sensor system (e.g., analyte sensor system 8 and/or analyte sensor system 208). In some embodiments, analyte sensor system 208 may be configured to measure analyte levels and inform a patient about the identification and/or prediction of adverse events, such as hyperglycemia and hypoglycemia. Additionally, the analyte sensor system 208 may be configured to help inform the type of guidance provided to the patient in response to these adverse events.

For example, the analyte sensor system 208 of FIG. 5 may be worn by a patient and configured to continuously measure the analyte levels of the patient over time using a continuous analyte sensor, such as the analyte sensor 530. The measured analyte levels may then be processed by the analyte sensor system 208 (e.g., by the processor/microcontroller 535) to identify and/or predict adverse events, and/or to provide guidance to the patient for treatment and or actions to abate or prevent the occurrence of such adverse events. Analyte data indicating the patient's analyte levels may then be output to the transceiver 510 of the analyte sensor system for wireless transmission to a communications device via the antenna system 545. In some cases, this information may be wirelessly communicated using a wireless communication link and antennas included in the analyte sensor system. In some embodiments, the wireless communication link may comprise a Bluetooth low energy (BLE) communication link, a WiFi communication link, a cellular communication link (e.g., 2G, 3G, 4G, 5G, and/or a future generation), or another type of wireless communication based on any other wireless communication technology described herein. In some embodiments, the communications device may be at least one of the display devices 210, the partner devices 215, or server system 234 illustrated and described with respect to FIG. 2.

In some embodiments, the antenna system 545 of the analyte sensor system 208 may have a particular radiation pattern. This radiation pattern may represent a relative power radiated by an antenna as a function of a spatial direction away from the antenna. The radiation pattern of the antenna system 545 may be determined based on physical characteristics of the one or more antennas of the antenna system 545, such as size, shape, and orientation, as well as an operating frequency. Power may be radiated by the one or more antennas of the antenna system 545 in a number of lobes or region of the radiation pattern that are bounded by points of equal radiation intensity or power.

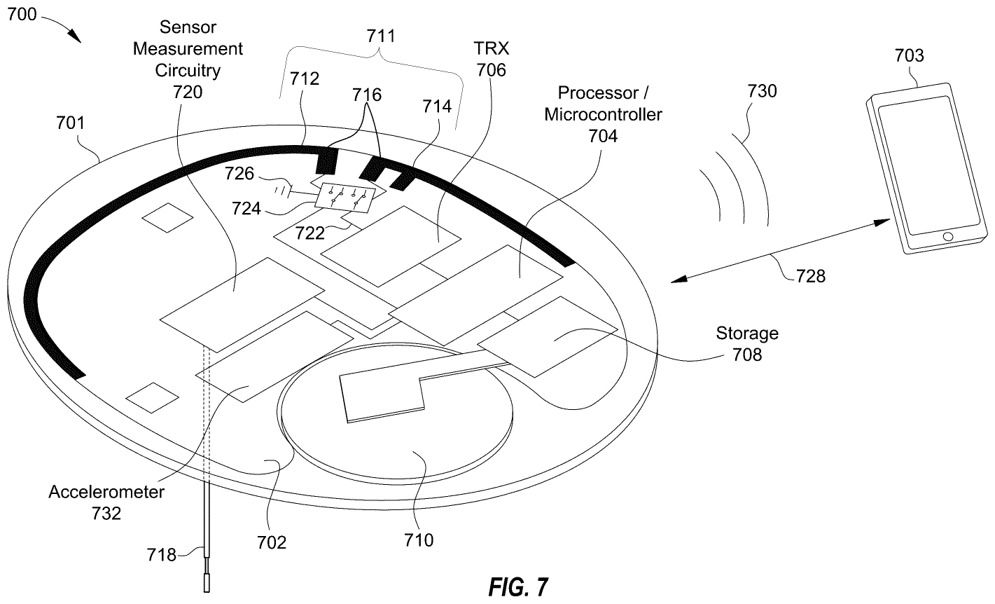

FIG. 6 illustrates an example radiation pattern 600 of the one or more antennas of the antenna system 545. As shown, the radiation pattern 600 includes a main lobe 602, a plurality of side lobes 604, and a back lobe 606. As shown, the main lobe 602 of the radiation pattern 600 represents the spatial direction of maximum radiation intensity or power. In other words, the main lobe 602 is the region of the radiation pattern where most of the radiated energy is concentrated. The main lobe 602 is generally considered to be the most important part of the radiation pattern, as it determines the spatial direction of maximum signal strength and is the spatial direction in which the antenna is pointing. In contrast, the plurality of side lobes 604 are regions of the radiation pattern where radiation intensity is lower than that of the main lobe 602. The plurality of side lobes 604 may occur when the one or more antennas of the antenna system 545 radiates power in spatial directions other than an intended spatial direction. As shown, the plurality of side lobes 604 may occur on either side of the main lobe 602. Similarly, the back lobe 606 is a type of side lobe consisting of regions of the radiation pattern directly opposite the main lobe 602. Both the plurality of side lobes 604 and the back lobe 606 are undesirable as have the potential to cause interference and decrease the overall efficiency of the one or more antennas of the antenna system 545 since power is being radiated in undesirable or un-useful spatial directions.

In some cases, certain existing analyte sensor systems may have a thickness of about 7 millimeters (mm), allowing one or more antennas of these analyte sensor systems to be located at a distance of about 5.5 mm away from a body of a user or patient due to certain design constraints. However, there is a constant competitive drive to miniaturize these analyte sensor systems, for example, to provide better comfort, discreet usage, and/or ease of use to the user. As a result, next-generation analyte sensor systems may be designed to have a thickness of less than half of the current analyte sensor systems. For example, these next-generation analyte sensor systems will have a thickness of about 2.9 mm, reducing the distance between the one or more antennas of these analyte sensor systems and the body of the user to approximately 2.2 mm. Moreover, these next-generation analyte sensor systems may be expected to have a longer communication range than current analyte sensor systems. For example, current analyte sensor systems may support a communication range of about 20 feet while next generation analyte sensor systems are expected to support a range of 30 feet or more. In some ideal scenarios, a larger power source (e.g., a larger battery) may provide a higher power communication (with a longer communication range), however, with miniaturization of the next-generation analyte sensor systems such implementations may be prohibitive.

The miniaturization of next generation analyte sensor systems and resulting reduction in distance between the one or more antennas and the body of the user may cause issues in achieving this expected increased communication range. Moreover, the reduction in distance between the one or more antennas and the body of the user may also cause issues related to a bandwidth associated with the sensor antenna. For example, as the one or more antennas are positioned closer to the body of the user, power radiated from a back lobe of the one or more antennas may be absorbed by the body of the user, causing a variation of input impedance, frequency shift and reduced efficiency and gain of the one or more antennas. As a result, due to the miniaturization of next-generation analyte sensor systems, the body of the user may negatively affect a radiation pattern of the one or more antennas of these analyte sensor systems, causing a reduction in communication range between these analyte sensor systems and associated communications devices and leading to poor user experience.

In some cases, one manner of reducing the negative effects associated with miniaturization and antennas located closer to a body of the user may be to use a larger antenna. For example, for some portable computational devices (e.g., smartphones, tablets, and laptop computers), the size of the device may allow for half-wave antennas to be used for wireless communications, which may improve antenna gain, antenna efficiency, frequency response, transmission range, etc. However, the size of next-generation analyte sensor systems may not allow these analyte sensor systems to be equipped with a large antenna (e.g., a half-wave antenna or larger). Instead, the size of these analyte sensor systems may only allow the use of quarter-wave antennas or smaller, which may have lower antenna gain and lower antenna performance and, thus, may not be able to transmit as far as larger antennas. In some cases, rather than using a larger antenna, a radiation pattern of the smaller, quarter-wave antenna may be designed to be directive, allowing radiated power to be concentrated in a particular spatial direction (e.g., a radiation pattern having a main lobe concentrated in a single direction) and improving antenna gain and/or performance of the quarter-wave antenna.

However, while directionality may improve antenna gain and/or performance, directionality may give rise to additional issues. For example, in some cases, the user of an analyte sensor system may continuously move locations throughout the day. These changes in location of the user (as well as the analyte sensor system) may change the direction of the radiation pattern (e.g., in particular the main lobe) of the antenna of the analyte sensor system relative to a communications device, leading to misalignment between the analyte sensor system and communications device. In some cases, these changes in position and misalignment between the analyte sensor system and the communications device may, in turn, lead to obstructions being positioned between the antenna of the sensor system and the communications device, such as the body of the user, another person, a wall, etc. Further, the misalignment and obstructions between the analyte sensor system and communications device may result in degraded communication performance (e.g., reduced throughput, increased latency, reduced transmission range, and/or a lost communication link). In addition to changes in the location of the user, the analyte sensor system may be placed on various different positions on the body of the user (e.g., arm, torso, leg), each of which may be associated with a different antenna efficiency and/or frequency response, causing similar issues with degraded communication performance over time.

Accordingly, aspects of the present disclosure provide techniques for improving antenna performance of a next-generation analyte sensor system, such as an analyte sensor system that is designed to have a small distance (e.g., below a threshold, such as approximately 5 mm) between one or more antennas included therein and a body of the user. In some embodiments, these techniques may involve equipping the analyte sensor system with a spatially diverse antenna architecture to enhance wireless communication performance of the analyte sensor system. The antenna architecture may include a plurality of antennas coupled to a switching device, such as a single pole, double throw switch. In some embodiments, each of the plurality of antennas may be quarter-wave antennas or smaller, allowing the plurality of antennas to fit within the (smaller) analyte sensor system. Additionally, the plurality of antennas may be arranged in different positions within the analyte sensor system to facilitate spatial diversity in radiation patterns of the analyte sensor system. For example, the plurality of antennas may include a first antenna, which may provide a radiation pattern oriented in a first spatial direction with a high directivity to facilitate an increased transmission range within the first spatial direction. Additionally, the plurality of antennas may include a second antenna, which may provide a radiation pattern oriented in a second spatial direction (different from the first spatial direction) with a high directivity to facilitate an increased transmission range in the second spatial direction. In some embodiments, the analyte sensor system may also include a third antenna formed through combination of the first antenna and the second antenna, which may provide a pseudo or near omni-directional radiation pattern, as explained below.

In some embodiments, the switching device may allow for selective coupling of one or more antennas to an antenna feed of a transceiver (e.g., transceiver 510 illustrated and described with respect to FIG. 5). In some embodiments, the switching device may be integrated with the transceiver of the analyte sensor system (e.g., integrated in a transceiver chip) or a discrete integrated circuit. In some cases, the switching device may couple multiple antennas together to effectively form a larger third antenna, such as an antenna having a dimension greater than a quarter-wave. Further, the combined antennas that form the larger third antenna may form a radiation pattern that provides a broader beam shape, such as a pseudo or near omni-directional radiation pattern. In some cases, the pseudo or near omni-directional radiation pattern may improve efficiency and/or the radiation energy, enabling communications with the communications device when the directivity of the individual antennas provides inferior channel conditions compared to the omni-directional radiation pattern of the antenna combination.

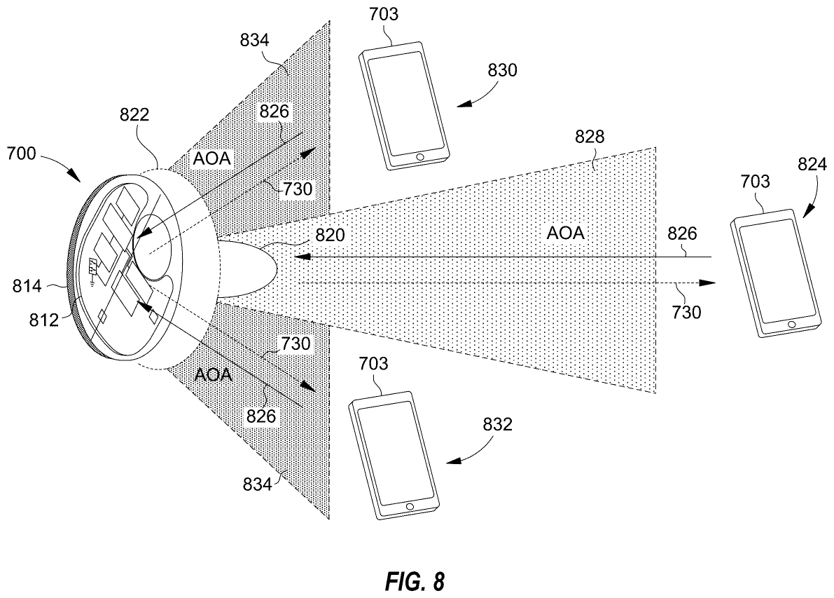

FIG. 7 illustrates a perspective view of an analyte sensor system 700 that includes an antenna system comprising a plurality of antennas that may be used to provide the analyte sensor system 700 with spatial diversity when transmitting analyte data associated with analyte levels of a user of the analyte sensor system 700 to a communications device 703. The analyte sensor system 700 may be an example of the analyte sensor system 208 depicted and described with respect to FIG. 2 and FIG. 5. As shown, the analyte sensor system 700 includes a waterproof housing 701 that may be adhered to a body of a user using, for example, an adhesive patch. The waterproof housing 701 may house one or more components of the analyte sensor system 700, including a printed circuit board (PCB) 702, a processor/microcontroller 704 (e.g., including one or more processors), a transceiver circuit 706 coupled to a plurality of antennas of an antenna system 711, a storage 708 (e.g., including one or more memories), a battery 710, an analyte sensor 718, sensor measurement circuitry 720, and an accelerometer 732. In some embodiments, the battery 710 may be configured to power the one or more components of the analyte sensor system 700. The accelerometer 732 may be configured to provide orientation information associated with the analyte sensor system 700.

As shown, the plurality of antennas of the antenna system 711 may include a first antenna 712, a second antenna 714, and a third antenna 716. In the embodiment shown in FIG. 7, the first antenna 712 and the second antenna 714 may each comprise a respective trace antenna arranged along an edge of the PCB 702. For example, as shown in FIG. 7, the first antenna 712 may comprise comprises an inverted-L antenna disposed along a first segment of an edge of the PCB 702 and the second antenna 714 may comprise a planar inverted-F antenna disposed along a second segment of the edge of the PCB 702. However, the first antenna 712 and the second antenna 714 may include any suitable antenna structure, for example, a patch antenna, a slot antenna, a trace antenna, a spiral antenna, a stamp antenna, an inverted-F antenna (including a planar inverted-F antenna (PIFA) and/or a meandered inverted-F antenna (MIFA)), an inverted-L antenna, a quarter-wave monopole, etc. As will be described in greater detail below, the third antenna 716 may be a combination of the first antenna 712 and the second antenna 714.

In some embodiments, the PCB 702 may include circuitry for operatively connecting the processor/microcontroller 704, the transceiver circuit 706, the plurality of antennas (e.g., the first antenna 712, the second antenna 714, and the third antenna 716) of the antenna system 711, the storage 708, the analyte sensor 718, and the sensor measurement circuitry 720. In some cases, the processor/microcontroller 704 may be an example of the processor/microcontroller 535 described with respect to FIG. 5, the transceiver circuit 706 may be an example of the transceiver 510 described with respect to FIG. 5, the antenna system 711 may be an example of the antenna system 545 described with respect to FIG. 5, the storage 708 may be an example of the storage 515 described with respect to FIG. 5, the analyte sensor 718 may be an example of the analyte sensor 530 described with respect to FIG. 5, and the sensor measurement circuitry 720 may be an example of the sensor measurement circuitry 525 described with respect to FIG. 5.

As noted above, the analyte sensor system 700 includes the analyte sensor 718 coupled with sensor measurement circuitry 720, which are configured to generate analyte data associated with analyte levels of the user of the analyte sensor system 700 and provide the analyte data to the processor/microcontroller 704. In some embodiments, the processor/microcontroller 704 may be configured to process the analyte data and provide the processed analyte data to the transceiver circuit 706 for transmission. For example, in some embodiments, after receiving the analyte data from the processor/microcontroller 704, the transceiver circuit 706 may output the analyte data to an antenna feed 722 of the transceiver circuit 706 for wireless transmission 730 to the communications device 703 via at least one of the first antenna 712, the second antenna 714, or the third antenna 716 of the antenna system 711.

In some embodiments, the communications device 703 may be a display device for displaying the analyte data to the user. In some cases, the communications device 703 may be an example of one or more of the display devices 210, the partner devices 215, and/or the server system 234 illustrated and described with respect to FIG. 2. In some embodiments, the communications device 703 may include a second antenna system, comprising one or more antennas, configured to receive the analyte data from the antenna system 711 of the analyte sensor system 700. The communications device 703 may be configured to display the analyte data received from the antenna system 711 of the analyte sensor system 700 to the user. In some embodiments, the antenna system 711 may be configured to transmit raw sensor data from the analyte sensor 718 to the communications device 703. In such cases, the communications device 703 may be configured to process the raw sensor data to obtain analyte levels of the user and to display the analyte levels to the user.

In some embodiments, the first antenna 712, the second antenna 714, and the third antenna 716 of the antenna system 711 may help to provide spatial diversity to the analyte sensor system 700 and improve a transmission/communication range 728 of wireless transmissions 730 between the analyte sensor system 700 and the communications device 703. For example, in some embodiments, the first antenna 712 may be arranged to have a first radiation pattern oriented with high directivity in a first spatial direction (e.g., azimuth and/or elevation) to facilitate an increased transmission range within the first spatial direction. Similarly, the second antenna 714 may be arranged to have a second radiation pattern oriented with high directivity in a second spatial direction to facilitate an increased transmission range in a second spatial direction. Additionally, in some embodiments, the third antenna 716 may be arranged to have a third radiation pattern, such as a pseudo or near omni-directional radiation pattern, which may facilitate an increased transmission range when the highly directive first radiation pattern and second radiation pattern associated with the first antenna 712 and second antenna 714 provide poor channel conditions for the analyte sensor system 700. Additional details regarding the selection and use of the first antenna 712, the second antenna 714, and the third antenna 716 will be presented below.

In some embodiments, the first antenna 712 and the second antenna 714 may have a dimension that is less than or equal to a quarter-wave of a transmission frequency or frequency band used for communications between the analyte sensor system 700 and the communications device 703, such as 2.4 GHz used for Bluetooth communication or other transmission frequencies used for other types of communication (e.g., WiFi communication, cellular communication (e.g., 2G, 3G, 4G, 5G, or later generations), etc.). Accordingly, in some embodiments, the first antenna 712 and the second antenna 714 may be quarter-wave antennas. In some embodiments, the first antenna 712 may have a first antenna structure (e.g., an inverted-L antenna), and the second antenna 714 may have a second antenna structure different from the first antenna 712. For example, the second antenna structure may be an inverted-F antenna, such as a planar inverted-F antenna (PIFA) and/or a meandered inverted-F antenna (MIFA). In some embodiments, the first antenna 712 and the second antenna 714 may have different polarizations. For example, the first antenna 712 may have a horizontal polarization while the second antenna 714 may have a vertical polarization.

In some embodiments, the third antenna 716 may be formed through a combination of at least the first antenna 712 and the second antenna 714. To facilitate this combination of the first antenna 712 and the second antenna 714, the analyte sensor system 700 further includes a switching device 724 that may be configured to couple the first antenna 712 with the second antenna 714 to form the third antenna 716. In some embodiments, the switching device 724 may include a single pole double throw (SPDT) switch. In some embodiments, the switching device 724 may be integrated with other circuitry on the PCB 702, such as a radio frequency integrated circuit, or a discrete chip. The third antenna 716 may be formed by coupling the first antenna 712 and the second antenna 714 in different manners. For example, in some embodiments, the third antenna 716 may be arranged such that the first antenna 712 and the second antenna 714 are coupled in parallel with the antenna feed 722 as depicted in FIG. 7. In certain embodiments, the third antenna 716 may be arranged such that the first antenna 712 is coupled in series with the second antenna 714, and the antenna feed 722 may be selectively coupled to either of the first antenna 712 or the second antenna 714.

In some embodiments, due to the combination of the first antenna 712 and the second antenna 714, the third antenna 716 may have a dimension that is greater than a quarter-wave of the transmission frequency or frequency band used for communications between the analyte sensor system 700 and the communications device 703. For example, in some embodiments, the third antenna 716 may effectively be a half-wave antenna. Further, in some embodiments, the third antenna 716 may have a combination of polarizations, for example, due to the different polarizations of the first antenna 712 and the second antenna 714.

In some embodiments, one or more processors of the analyte sensor system 700, such as the processor/microcontroller 704, may be configured to cause the analyte sensor system 700 to perform various actions described below associated with selectively coupling one or more antennas of the antenna system 711 to the transceiver circuit 706 for communicating information (e.g., analyte data or other types of information described herein) to a communications device. For example, in some cases, the processor/microcontroller 704 may be configured to cause the analyte sensor system to use the switching device 724 to selectively couple any of the first antenna 712, the second antenna 714, or the third antenna 716 of the antenna system 711 to the antenna feed 722 of the transceiver circuit 706 and/or a ground terminal 726 based on one or more criteria. In some cases, the one or more criteria may involve channel conditions between the analyte sensor system 700 and the communications device 703 for each respective antenna of the plurality of antennas of the antenna system 711 analyte sensor system 700.

For example, in some embodiments, the processor/microcontroller 704 may be configured to cause the analyte sensor system 700 to monitor channel conditions between the analyte sensor system 700 and the communications device 703 via the plurality of antennas of the antenna system 711 of the analyte sensor system 700 and to select an antenna (e.g., the first antenna 712 or the second antenna 714) or an antenna combination (e.g., the third antenna 716) of the plurality of antennas of the antenna system 711 that provides the best channel conditions. In some embodiments, the best channel conditions may comprise channel conditions having a highest signal quality, a highest signal strength, a lowest data error rate or ratio, a highest throughput, and the like for the wireless transmissions 730 to the communications device 703.

As an example, the processor/microcontroller 704 may be configured to cause the analyte sensor system 700 to select an antenna of the plurality of antennas of the antenna system 711 that provides the strongest signal strength for the wireless transmissions 730 between the analyte sensor system 700 and the communications device 703. In some embodiments, the processor/microcontroller 704 may be configured to cause the analyte sensor system 700 to determine which antenna of the plurality of antennas of the antenna system 711 has the best channel conditions in different manners. For example, in some embodiments, the analyte sensor system 700 may transmit a pre-defined signal (e.g., a pilot signal or known data transmission) to the communications device 703 via one or more of the first antenna 712, the second antenna 714, and/or the third antenna 716. In response to the transmitted signals, the processor/microcontroller 704 may be configured to cause the analyte sensor system 700 to receive, from the communications device 703, feedback associated with the channel conditions.

The feedback may indicate the channel conditions associated with one or more of the antennas based on the transmissions received at the communications device 703. For example, the feedback may include a received signal strength indication (RSSI) (or any other suitable parameter indicative of the channel conditions) as observed at the communications device 703 for the signals transmitted via each of the plurality of antennas of the antenna system 711 of the analyte sensor system 700. For example, in some embodiments, the feedback received from the communications device 703 may include a first RSSI value associated with the first antenna 712, a second RSSI value associated with the second antenna 714, and a third RSSI value associated with the third antenna 716. The processor/microcontroller 704 may be configured to cause the analyte sensor system 700 to then select the antenna (e.g., the first antenna 712 or the second antenna 714) or antenna combination (e.g., the third antenna 716) for transmitting the wireless transmissions 730 to the communications device 703 based on the feedback. In some cases, these wireless transmissions 730 may include analyte data of a user of the analyte sensor system 700 or other information exchanged between the analyte sensor system 700 and the communications device 703.

In some cases, the processor/microcontroller 704 may be configured to cause the analyte sensor system 700 to select the antenna of the plurality of antennas of the antenna system 711 having the strongest RSSI among the first RSSI value for the first antenna 712, the second RSSI value for the second antenna 714, and the third RSSI value for the third antenna 716. For example, in some embodiments, the processor/microcontroller 704 may be configured to cause the analyte sensor system 700 to select the first antenna 712 to couple to the antenna feed 722 via the switching device 724 for transmitting the analyte data when the first RSSI value for the first antenna 712 is greater than both the second RSSI value for the second antenna 714 and the third RSSI value for the third antenna 716. In some embodiments, the processor/microcontroller 704 may be configured to cause the analyte sensor system 700 to select the second antenna 714 to couple to the antenna feed 722 via the switching device 724 for transmitting the analyte data when the second RSSI value for the second antenna 714 is greater than both the first RSSI value for the first antenna 712 and the third RSSI value for the third antenna 716. In some embodiments, the processor/microcontroller 704 may be configured to cause the analyte sensor system 700 to select the third antenna 716 to couple to the antenna feed 722 via the switching device 724 for transmitting the analyte data when the third RSSI value for the third antenna 716 is greater than both the first RSSI value for the first antenna 712 and the second RSSI value for the second antenna 714.

In some embodiments, the processor/microcontroller 704 may be configured to cause the analyte sensor system 700 to select the antenna of the plurality of antennas of the antenna system 711 having an RSSI greater than or equal to a threshold. The threshold may be a configurable value. In certain aspects, a default value for the threshold may be determined based on a calibration process that identifies the RSSI threshold associated with a specified communication performance, such as a particular packet error rate (PER), data rate, latency, etc. For example, the calibration process may determine the RSSI that corresponds to a packet error rate of 30% for various transmission ranges and/or channel conditions (e.g., reflections or line of sight). In some cases, the threshold may represent the lowest signal strength that can be used for reliable wireless communications between the analyte sensor system 700 and the communications device 703.

As an example, if the communications device 703 has a receiver sensitivity of −95 dBm, the threshold may be set to −92 dBm±3 dBm to ensure reliable wireless communications (e.g., low signal loss). In some embodiments, the processor/microcontroller 704 may be configured to cause the analyte sensor system 700 to perform the antenna selection periodically or in response to certain criteria, such as a change in performance with the current selected antenna of the plurality of antennas of the antenna system 711. For example, the processor/microcontroller 704 may be configured to cause the analyte sensor system 700 to use a particular antenna of the plurality of antennas of the antenna system 711 for as long as an RSSI (or a time-averaged RSSI) for that antenna remains above an RSSI threshold. In response to detecting that the channel conditions for the antenna indicate to switch to a different antenna of the plurality of antennas of the antenna system 711 (e.g., when the RSSI or time-averaged RSSI is below the RSSI threshold), the processor/microcontroller 704 may be configured to cause the analyte sensor system 700 to select a different antenna of the plurality of antennas of the antenna system 711 to use for the wireless transmissions 730 to the communications device 703.

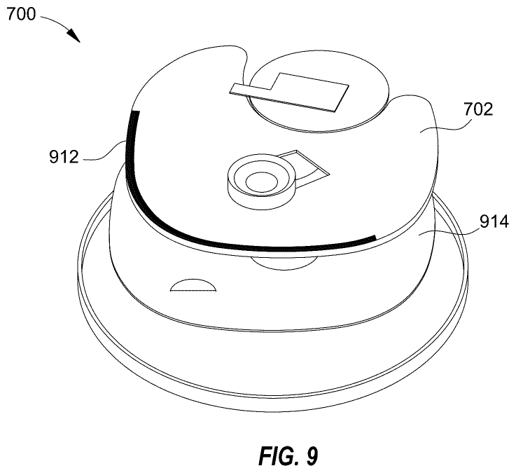

In some embodiments, the processor/microcontroller 704 may be configured to cause the analyte sensor system 700 to select an antenna of the plurality of antennas of the antenna system 711 for transmitting the wireless transmissions 730 to the communications device 703 based on angle of arrival (AOA) information associated with transmissions received from the communications device 703. An example of these techniques for selecting an antenna based on AOA information is illustrated in FIG. 8. For example, FIG. 8 illustrates an example embodiment of the analyte sensor system 700 in which the first antenna 712 described with respect to FIG. 7 may be a trace antenna 812 and the second antenna 714 described with respect to FIG. 7 may be a stamp antenna 814. The trace antenna 812 may be designed to have a radiation pattern 820 with high directivity for line-of-sight (LOS) paring or communication while the stamp antenna 814 may be designed to have a wider, pseudo or near omni-directional radiation pattern 822 for non-LOS communication.

In some embodiments, whether the analyte sensor system 700 selects the trace antenna 812 (e.g., the first antenna 712), the stamp antenna 814 (e.g., the second antenna 714), or a combination of the trace antenna 812 and the stamp antenna 814 (e.g., the third antenna 716) may be based on AOA information associated with transmissions received from the communications device 703. For example, as shown in FIG. 8, when the communications device 703 is located at a first position as shown at 824, transmissions 826 received by the analyte sensor system 700 from the communications device 703 may be within an antenna radiation range 828 that is able to be served by the radiation pattern 820 of the trace antenna 812. The processor/microcontroller 704 may be configured to cause the analyte sensor system 700 to determine that the transmissions 826 received from the communications device 703 are within the antenna radiation range 828 associated with the trace antenna 812 based on AOA information associated with the transmissions 826, which may be determined by the analyte sensor system 700 using accelerometer 732 of the analyte sensor system 700 described with respect to FIG. 7 and RSSI associated with the transmissions 826.

For example, in some embodiments, the processor/microcontroller 704 may be configured to cause the analyte sensor system 700 to receive the transmissions 826 from the communications device 703, determine the AOA information associated with the transmissions 826 (e.g., using orientation information provided by the accelerometer 732), and determine an RSSI associated with the transmissions 826. The processor/microcontroller 704 may then cause the analyte sensor system 700 to determine, based on the AOA information and RSSI associated with the transmissions 826, that the transmission 826 and the communications device 703 are within the antenna radiation range 828 associated with the trace antenna 812. Accordingly, based on the determination that the communications device 703 is within the antenna radiation range 828 associated with the trace antenna 812, the analyte sensor system 700 (e.g., via the processor/microcontroller 704) may select, using the switching device 724, the trace antenna 812 for communicating (e.g., transmitting the wireless transmissions 730 described with respect to FIG. 7) with the communications device 703. In this case, the analyte sensor system 700 may use the switching device 724 to couple the stamp antenna 814 to ground (e.g., ground terminal 726), allowing the analyte sensor system 700 to have a stable connection with the communications device 703 with maximum power.

In some cases, the processor/microcontroller 704 may be configured to cause the analyte sensor system 700 to instead select the stamp antenna 814 (e.g., having the wider, pseudo or near omni-directional radiation pattern 822) for communicating with the communications device 703 when the transmissions 826 and, thereby the communications device 703, are outside the antenna radiation range 828 associated with the trace antenna 812. For example, when the communications device 703 is located at a second position, as shown at 830 or 832, the processor/microcontroller 704 may be configured to cause the analyte sensor system 700 to receive the transmissions 826 from the communications device 703, determine the AOA information associated with the transmissions 826 (e.g., using the orientation information from the accelerometer 732), and determine the RSSI associated with the transmissions 826. The processor/microcontroller 704 may then cause the analyte sensor system 700 to determine, based on the AOA information and RSSI associated with the transmissions 826, that the transmission 826 and the communications device 703 are not within the antenna radiation range 828 associated with the trace antenna 812 but are instead within an antenna radiation range 834 served by the wider, pseudo or near omni-directional radiation pattern 822 of the stamp antenna 814.

Accordingly, based on the determination that the communications device 703 is within the antenna radiation range 834 associated with the stamp antenna 814, the analyte sensor system 700 (e.g., via the processor/microcontroller 704) may select, using the switching device 724, the stamp antenna 814 for communicating (e.g., transmitting the wireless transmissions 730 described with respect to FIG. 7) with the communications device 703. In this case, the analyte sensor system 700 may use the switching device 724 to couple the trace antenna 812 to ground (e.g., ground terminal 726), allowing the analyte sensor system 700 to have a stable connection with the communications device 703 with maximum power.

In some cases, the AOA information and RSSI may be used to direct the user of the analyte sensor system 700 to get closer to the communications device 703 in a scenario in which neither the first antenna 712, the second antenna 714, nor the third antenna 716 are able to achieve channel conditions (e.g., RSSI) above a threshold between the analyte sensor system 700 and the communications device 703.

In some embodiments, the switching device 724 may allow the analyte sensor system 700 to switch antennas when the communications device 703 moves positions. For example, in some embodiments, the communications device 703 may receive analyte data from the analyte sensor system 700 in the first position, as shown at 824. In some embodiments, the analyte sensor system 700 may be configured to transmit the analyte data to the communications device 703 in the first position using the trace antenna 812 (e.g., the first antenna 712). Thereafter, in some cases, the communications device 703 may move to the second position different from the first position, as shown at 830 or 832. In some cases, the communications device 703 to fail to receive the analyte data from the analyte sensor system 700 in the second position. In some embodiments, the analyte sensor system 700 may use the switching device to switch to the stamp antenna 814 (e.g., second antenna 714). In some embodiments, the analyte sensor system 700 may switch to the stamp antenna based on AOA information and/or RSSI, as described above. In some embodiments, based on the analyte sensor system 700 switching the antenna, the communications device 703 may then receive the analyte data from the analyte sensor system 700 in the second position.

In some embodiments, the switching device 724 may allow the analyte sensor system 700 to use the first antenna 712 and the second antenna 714 in different manners to enhance antenna operation and increase a transmission range of the analyte sensor system 700. For example, the switching device 724 may allow the analyte sensor system 700 to use the first antenna 712 and the second antenna 714 as a ground plane, exciter (directing element or director), reflector (reflecting element), and/or radiator. Using the first antenna 712 and the second antenna 714 in these different manners may allow the analyte sensor system 700 to further control the radiation pattern emitted from an active antenna.

For example, in some embodiments, the switching device 724 may allow the analyte sensor system 700 to selectively couple an inactive (e.g., non-selected) antenna of the plurality of antennas of the antenna system 711 to a ground terminal (e.g., ground terminal 726 illustrated in FIG. 7) to adjust or control the ground plane associated with an active (e.g., selected) antenna. For example, when the first antenna 712 is selectively coupled to the antenna feed 722 of a transceiver circuit 706, the second antenna 714 may be selectively coupled to the ground terminal 726 to provide an additional or alternative ground plane for the first antenna 712 (e.g., quarter-wave antenna). In some aspects, using the second antenna 714 as an additional or alternative ground plane for the first antenna 712 (or vice versa) may allow the analyte sensor system 700 to adjust the directivity (e.g., azimuth and/or elevation) and/or radiation pattern (e.g., omni-directional, directional, beam shape, beam size, etc.) of the first antenna 712. Adjusting the directivity and/or radiation pattern of the first antenna 712 may increase a communication range associated with first antenna 712 and overall communication quality between the analyte sensor system 700 and the communication device 703.

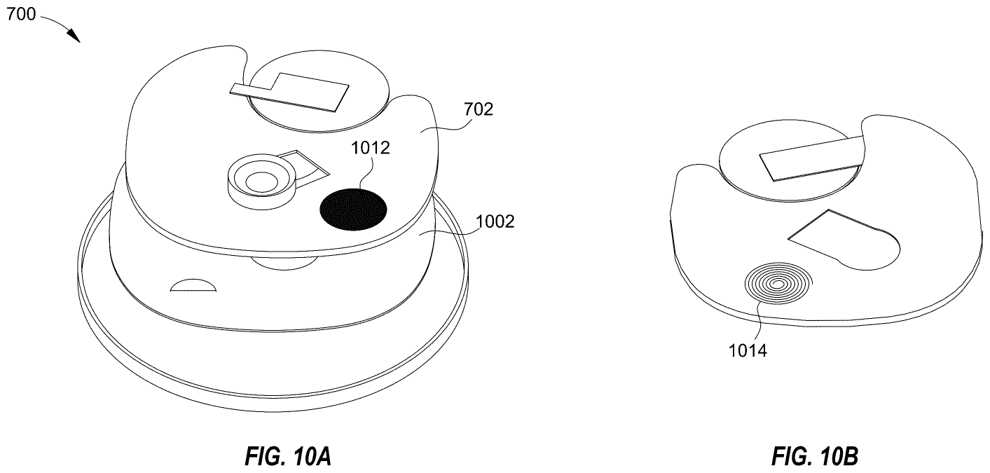

FIGS. 9 and 10 illustrate additional embodiments related to using the first antenna 712 and second antenna 714 of the antenna system 711 in different manners to enhance antenna operation and increase a transmission range of the analyte sensor system 700. For example, FIG. 9 illustrates an example embodiment of the analyte sensor system 700 in which the first antenna 712 described with respect to FIG. 7 is an on-board antenna 912 (e.g., located on the PCB 702), such as a trace antenna, and the second antenna 714 described with respect to FIG. 7 is an off-board antenna 914 (e.g., located off the PCB 702), such as a stamp antenna, a chip antenna, or another type of on-board antenna. As shown, on-board antenna 912 in FIG. 9 may be arranged along an edge of the PCB 702 of the analyte sensor system 700 while off-board antenna 914 is arranged below the PCB 702 and below the on-board antenna 912. In some cases, the on-board antenna 912 (e.g., first antenna 712) may be a different type of antenna as compared to the off-board antenna 914 (e.g., second antenna 714) such that the polarities of these antennas are different (e.g., opposite). Having different polarities may help to avoid coupling between these antennas, which would otherwise negatively affect a radiation pattern and transmission range of the antenna system.

Using the techniques described above, the processor/microcontroller 704 may be configured to cause the analyte sensor system 700 to use the switching device 724 described with respect to FIG. 7 to select among the on-board antenna 912 (e.g., the first antenna 712), the off-board antenna 914 (e.g., the second antenna 714), and a combination of the on-board antenna 912 and the off-board antenna 914 (e.g., the third antenna 716) as an active antenna for transmitting the wireless transmissions 730 to the communications device 703 described with respect to FIG. 7. In the example embodiment shown in FIG. 9, when the analyte sensor system 700 selects the off-board antenna 914 (e.g., the second antenna 714) to be the active antenna, the off-board antenna 914 may serve as a radiator. Additionally, in this case, the on-board antenna 912 may serve as an exciter or directing element to generate constructive interference and effectively adjust a directivity of a radiation pattern of the off-board antenna 914. In some cases, when the analyte sensor system 700 selects the on-board antenna 912 to be the active antenna, the on-board antenna 912 may serve as a radiator. Additionally, in this case, the off-board antenna 914 may be coupled (e.g., by switching device 724 illustrated in FIG. 7) to a ground terminal (e.g., ground terminal 726 described with respect to FIG. 7) and may serve as a reflector (e.g., a reflecting element) to reflect some of the radiation emitted from the on-board antenna 912 (e.g., a back lobe) in a particular direction, for example, to form a concentrated main lobe.

FIGS. 10A and 10B illustrate an example embodiment of the analyte sensor system 700 in which the first antenna 712 described with respect to FIG. 7 is a slot antenna 1012 and the second antenna 714 described with respect to FIG. 7 is a spiral antenna 1014. In some cases, the antenna architecture shown in FIGS. 10A and 10B may facilitate an omni-directional radiation pattern that may be tuned to be directional when the spiral antenna 1014 serves as a reflector or when the slot antenna 1012 serves as a directing element. The slot antenna 1012 and spiral antenna 1014 antenna architecture may facilitate improved radiation efficiency via the spatial diversity and directivity of the various antenna states. As shown in FIG. 10A, the slot antenna 1012 may be arranged on a top surface (or layer) of the PCB 702 and, as shown in FIG. 10B, the spiral antenna 1014 may be arranged on the bottom surface (or layer) of the PCB 702 below the slot antenna 1012. The spiral antenna 1014 may be arranged such that the slot antenna 1012 partially or fully overlaps with the spiral antenna 1014. The spiral antenna 1014 and the slot antenna 1012 may be arranged in stacked layout with PCB 702 arranged between the spiral antenna 1014 and the slot antenna 1012, such that the spiral antenna 1014 and the slot antenna 1012 are stacked on top of each other. The spiral antenna 1014 may be the same size as the slot antenna 1012, for example, having the same diameter, width, and/or height. Additionally, as shown in FIG. 10A, the analyte sensor system 700 may include a ground plane 1002 disposed beneath the PCB 702.

Using the techniques described above, the processor/microcontroller 704 may be configured to cause the analyte sensor system 700 to use the switching device 724 described with respect to FIG. 7 to select among the slot antenna 1012 (e.g., the first antenna 712), the spiral antenna 1014 (e.g., the second antenna 714), and a combination of the slot antenna 1012 and the spiral antenna 1014 (e.g., the third antenna 716) as an active antenna for transmitting the wireless transmissions 730 to the communications device 703 described with respect to FIG. 7. In the example embodiment shown in FIGS. 10A and 10B, when the analyte sensor system 700 selects the spiral antenna 1014 (e.g., the second antenna 714) to be the active antenna, the spiral antenna 1014 may serve as a radiator. Additionally, in this case, the ground plane 1002 may serve as a reflector to reflect energy radiated by the spiral antenna 1014 and the slot antenna 1012 may serve as an exciter or directing element to generate constructive interference and effectively adjust a directivity of a radiation pattern of the spiral antenna 1014. In some cases, when the analyte sensor system 700 selects the slot antenna 1012 to be the active antenna, the slot antenna 1012 may serve as the radiator. Additionally, in this case, the slot antenna 1012 may couple with the spiral antenna 1014 and the ground plane 1002 and may serve as a reflector (e.g., a reflecting element) to reflect some of the radiation emitted from the slot antenna 1012 (e.g., a back lobe) in a particular direction, for example, to form a concentrated main lobe.

### Example Operations

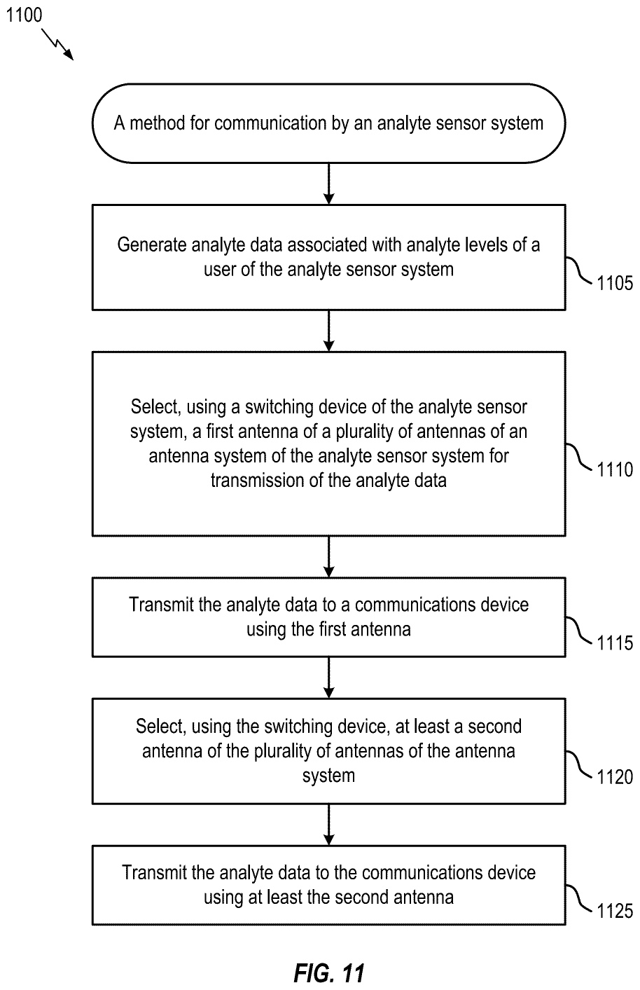

FIG. 11 shows a method 1100 for wireless communications by an analyte sensor system, such as the analyte sensor system 8 depicted and described with respect to FIG. 1, the analyte sensor system 208 depicted and described with respect to FIG. 2 and FIG. 5, and/or the analyte sensor system 700 depicted and described with respect to FIGS. 7, 8, 9, 10A, and 10B.

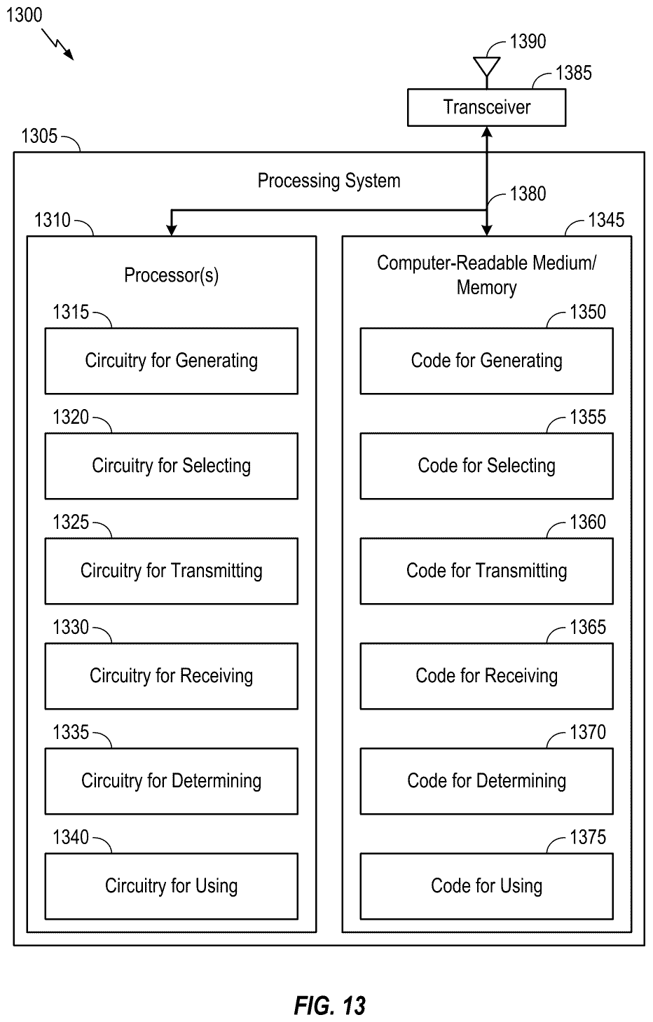

Method 1100 begins at step 1105 with generating analyte data associated with analyte levels of a user of the analyte sensor system. In some cases, the operations of this step refer to, or may be performed by, circuitry for generating and/or code for generating as described with reference to FIG. 13.

Method 1100 then proceeds to step 1110 with selecting, using a switching device of the analyte sensor system, a first antenna of a plurality of antennas of an antenna system of the analyte sensor system for transmission of the analyte data. In some cases, the operations of this step refer to, or may be performed by, circuitry for selecting and/or code for selecting as described with reference to FIG. 13.

Method 1100 then proceeds to step 1115 with transmitting the analyte data to a communications device using the first antenna. In some cases, the operations of this step refer to, or may be performed by, circuitry for transmitting and/or code for transmitting as described with reference to FIG. 13.

Method 1100 then proceeds to step 1120 with selecting, using the switching device, at least a second antenna of the plurality of antennas of the antenna system. In some cases, the operations of this step refer to, or may be performed by, circuitry for selecting and/or code for selecting as described with reference to FIG. 13.

Method 1100 then proceeds to step 1125 with transmitting the analyte data to the communications device using at least the second antenna. In some cases, the operations of this step refer to, or may be performed by, circuitry for transmitting and/or code for transmitting as described with reference to FIG. 13.

In some aspects, selecting, using the switching device, the first antenna and at least the second antenna is based at least in part on channel conditions associated with the plurality of antennas of the antenna system.

In some aspects, the method 1100 further includes receiving, from the communications device, feedback indicative of the channel conditions. In some cases, the operations of this step refer to, or may be performed by, circuitry for receiving and/or code for receiving as described with reference to FIG. 13.

In some aspects, the feedback includes a received signal strength indicator (RSSI) associated with each of the antennas of the antenna system.

In some aspects, the method 1100 further includes receiving one or more transmissions from the communications device. In some cases, the operations of this step refer to, or may be performed by, circuitry for receiving and/or code for receiving as described with reference to FIG. 13.

In some aspects, the method 1100 further includes determining angle-of-arrival (AOA) information associated with the one or more transmissions received from the communications device. In some cases, the operations of this step refer to, or may be performed by, circuitry for determining and/or code for determining as described with reference to FIG. 13.

In some aspects, determining the AOA information associated with the one or more transmissions received from the communications device is based, at least in part, on orientation information from an accelerometer of the analyte sensor system.

In some aspects, selecting, using the switching device, the first antenna and at least the second antenna is based at least in part on the AOA information associated with the one or more transmissions received from the communications device.

In some aspects, the first antenna is associated with a first radiation range; and the second antenna is associated with a second radiation range.

In some aspects, selecting the first antenna, using the switching device, comprises selecting the first antenna when, based on the AOA information, the one or more transmissions are determined to be received in the first radiation rage associated with the first antenna; and selecting the second antenna, using the switching device, comprises selecting the second antenna when, based on the AOA information, the one or more transmissions are determined to be received in the second radiation rage associated with the second antenna.

In some aspects, the method 1100 further includes using the switching device to couple the second antenna to a ground terminal when the first antenna is selected. In some cases, the operations of this step refer to, or may be performed by, circuitry for using and/or code for using as described with reference to FIG. 13.

In some aspects, the method 1100 further includes using the switching device to couple the first antenna to the ground terminal when the second antenna is selected. In some cases, the operations of this step refer to, or may be performed by, circuitry for using and/or code for using as described with reference to FIG. 13.

In some aspects, the plurality of antennas of the antenna system includes a third antenna; and the third antenna includes the first antenna selectively coupled to at least the second antenna.

In some aspects, the first antenna is disposed above a first surface of a circuit board of the analyte sensor system; the second antenna is disposed below a second surface of the circuit board; the first antenna is configured to operate as a directing antenna element when the third antenna is used to transmit a signal; and the second antenna is configured to operate as a reflecting antenna element when the first antenna is used to transmit a signal.

In some aspects, the first antenna comprises an inverted-L antenna disposed along a first segment of an edge of the circuit board; and the second antenna comprises a planar inverted-F antenna disposed along a second segment of the edge of the circuit board.

In some aspects, the first antenna comprises a trace antenna; and the second antenna comprises a stamp antenna.

In some aspects, the first antenna comprises a slot antenna; and the second antenna comprises a spiral antenna.

In some aspects, the plurality of antennas of the antenna system includes at least one of a patch antenna, a slot antenna, a trace antenna, a spiral antenna, a stamp antenna, an inverted-F antenna, or an inverted-L antenna.

In some aspects, the plurality of antennas of the antenna system includes at least one quarter-wave antenna configured to transmit a signal at an operating frequency of 2.4 GHz.

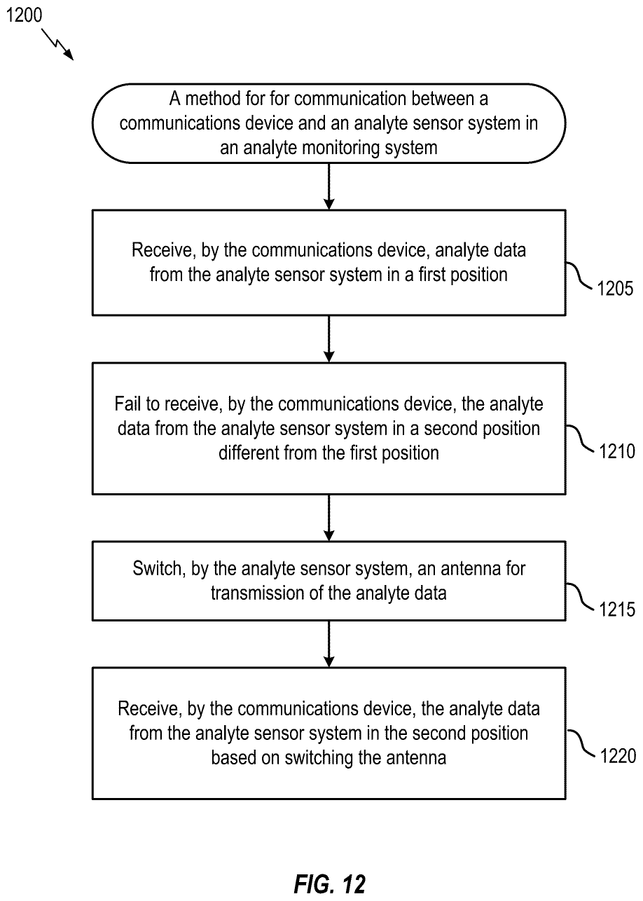

FIG. 12 shows a method for communication between a communications device and an analyte sensor system in an analyte monitoring system. In some embodiments, the analyte sensor system may be an example of the analyte sensor system 8 depicted and described with respect to FIG. 1, the analyte sensor system 208 depicted and described with respect to FIG. 2 and FIG. 5, and/or the analyte sensor system 700 depicted and described with respect to FIGS. 7, 8, 9, 10A, and 10B. In some embodiments, the communications device may be an example of the display devices 110, 120, 130, and 140, partner devices 136, and/or server system 134 depicted and described with respect to FIG. 1 and/or the display device 210, the partner device 215, or the server system 234 depicted and described with respect to FIG. 2.

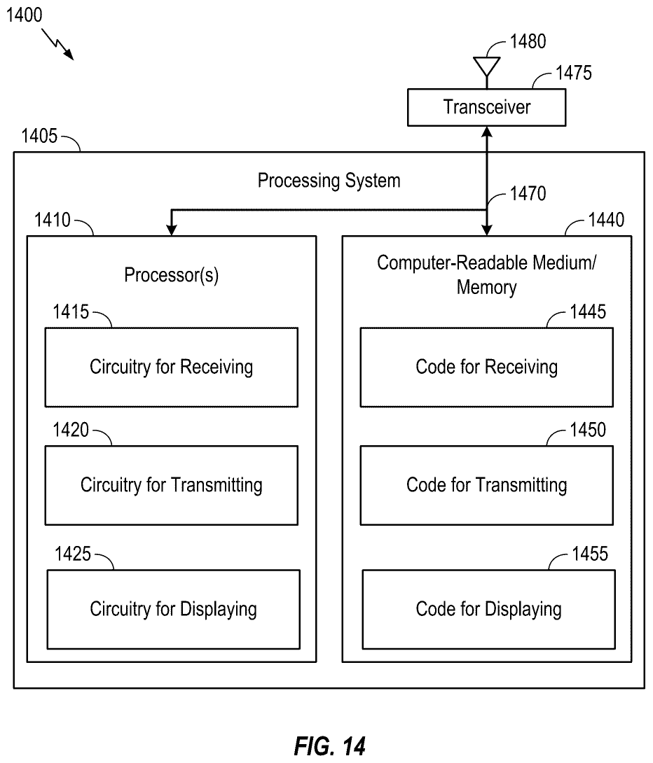

Method 1200 begins at step 1205 with receiving, by the communications device, analyte data from the analyte sensor system in a first position. In some cases, the operations of this step refer to, or may be performed by, circuitry for receiving and/or code for receiving as described with reference to FIG. 14.

Method 1200 then proceeds to step 1210 with failing to receive, by the communications device, the analyte data from the analyte sensor system in a second position different from the first position. In some cases, the operations of this step refer to, or may be performed by, circuitry for failing and/or code for failing as described with reference to FIG. 14.

Method 1200 then proceeds to step 1215 with switching, by the analyte sensor system, an antenna for transmission of the analyte data. In some cases, the operations of this step refer to, or may be performed by, circuitry for switching and/or code for switching as described with reference to FIG. 13.

Method 1200 then proceeds to step 1220 with receiving, by the communications device, the analyte data from the analyte sensor system in the second position based on switching the antenna. In some cases, the operations of this step refer to, or may be performed by, circuitry for receiving and/or code for receiving as described with reference to FIG. 14.

In some aspects, switching from the first antenna to the second antenna is based at least in part on channel conditions associated with the plurality of antennas of the antenna system.

In some aspects, the feedback includes a received signal strength indicator (RSSI) associated with each of the antennas of the antenna system.

In some aspects, determining the AOA information associated with the one or more transmissions received from the communications device is based, at least in part, on orientation information from an accelerometer of the analyte sensor system.

In some aspects, switching, by the analyte sensor system, from the first antenna to the second antenna is based at least in part on the AOA information associated with the one or more transmissions received from the communications device.

In some aspects, switching, by the analyte sensor system, from the first antenna to the second antenna comprises switching to the second antenna when, based on the AOA information, the one or more transmissions are determined, by the analyte sensor system, to be received in the second radiation rage associated with the second antenna.

In some aspects, the method 1200 further includes using, by the analyte sensor system, the switching device to couple the second antenna to a ground terminal when the first antenna is selected. In some cases, the operations of this step refer to, or may be performed by, circuitry for using and/or code for using as described with reference to FIG. 13.

In some aspects, the method 1200 further includes using, by the analyte sensor system, the switching device to couple the first antenna to the ground terminal when the second antenna is selected. In some cases, the operations of this step refer to, or may be performed by, circuitry for using and/or code for using as described with reference to FIG. 13.

In some aspects, the first antenna is disposed above a first surface of a circuit board of the analyte sensor system; the second antenna is disposed below a second surface of the circuit board; the first antenna is configured to operate as a directing antenna element when the third antenna is used to transmit a signal; and the second antenna is configured to operate as a reflecting antenna element when the first antenna is used to transmit a signal.

In some aspects, the first antenna comprises an inverted-L antenna disposed along a first segment of an edge of the circuit board; and the second antenna comprises a planar inverted-F antenna disposed along a second segment of the edge of the circuit board.

In some aspects, the first antenna comprises a trace antenna; and the second antenna comprises a stamp antenna.

In some aspects, the first antenna comprises a slot antenna; and the second antenna comprises a spiral antenna.

In some aspects, the first antenna is associated with a first radiation range; and the second antenna is associated with a second radiation range.

In some aspects, the method 1200 further includes switching, by the analyte sensor system, from the second antenna to the first antenna when, based on the AOA information, the one or more transmissions are determined, by the analyte sensor system, to be received in the first radiation rage associated with the first antenna. In some cases, the operations of this step refer to, or may be performed by, circuitry for switching and/or code for switching as described with reference to FIG. 13.

### Example Health Monitoring Devices

FIG. 13 depicts aspects of an example health monitoring device 1300. In some aspects, health monitoring device 1300 is an analyte sensor system, such as the analyte sensor system 8 described with respect to FIGS. 1, the analyte sensor system 208 of FIGS. 2 and 5, and/or the analyte sensor system 700 of FIGS. 7, 8, 9, 10A, and 10B.

The health monitoring device 1300 includes a processing system 1305 coupled to the transceiver 1385 (e.g., a transmitter and/or a receiver). The transceiver 1385 is configured to transmit and receive signals for the health monitoring device 1300 via the first antenna system 1390, such as the various signals and messages as described herein. The processing system 1305 may be configured to perform processing functions for the health monitoring device 1300, including processing signals received and/or to be transmitted by the health monitoring device 1300.

The processing system 1305 includes one or more processors 1310. In various aspects, the one or more processors 1310 may be representative of the processor/microcontroller 535, as described with respect to FIG. 5. The one or more processors 1310 are coupled to a computer-readable medium/memory 1345 via a bus 1380. In some aspects, the computer-readable medium/memory 1345 may be representative of the storage 515, as described with respect to FIG. 2. In certain aspects, the computer-readable medium/memory 1345 is configured to store instructions (e.g., computer-executable code) that when executed by the one or more processors 1310, cause the one or more processors 1310 to perform the methods 1100 and/or 1200 described with respect to FIGS. 11 and 12, or any aspects related to these methods. Note that reference to a processor performing a function of health monitoring device 1300 may include one or more processors 1310 performing that function of health monitoring device 1300.

In the depicted example, computer-readable medium/memory 1345 stores code (e.g., executable instructions), such as code for generating 1350, code for selecting 1355, code for transmitting 1360, code for receiving 1365, code for determining 1370, and code for using 1375. Processing of the code for generating 1350, code for selecting 1355, code for transmitting 1360, code for receiving 1365, code for determining 1370, and code for using 1375 may cause the health monitoring device 1300 to perform the methods 1100 and/or 1200 described with respect to FIGS. 11 and 12, or any aspects related to these methods.

The one or more processors 1310 include circuitry configured to implement (e.g., execute) the code stored in the computer-readable medium/memory 1345, including circuitry for generating 1315, circuitry for selecting 1320, circuitry for transmitting 1325, circuitry for receiving 1330, circuitry for determining 1335, and circuitry for using 1340. Processing with circuitry for generating 1315, circuitry for selecting 1320, circuitry for transmitting 1325, circuitry for receiving 1330, circuitry for determining 1335, and circuitry for using 1340 may cause the health monitoring device 1500 to perform the methods 1100 and/or 1200 described with respect to FIGS. 11 and 12, or any aspects related to these methods.

FIG. 14 depicts aspects of an example health monitoring device 1400. In some aspects, health monitoring device 1400 is a communications device, such as display devices 110, 120, 130, and 140, partner devices 136, and/or server system 134 depicted and described with respect to FIG. 1 and/or the display device 210, the partner device 215, or the server system 234 depicted and described with respect to FIG. 2.

The health monitoring device 1400 includes a processing system 1405 coupled to the transceiver 1475 (e.g., a transmitter and/or a receiver). The transceiver 1475 is configured to transmit and receive signals for the health monitoring device 1400 via the second antenna system 1480, such as the various signals and messages as described herein. The processing system 1405 may be configured to perform processing functions for the health monitoring device 1400, including processing signals received and/or to be transmitted by the health monitoring device 1400.

The processing system 1405 includes one or more processors 1410. The one or more processors 1410 are coupled to a computer-readable medium/memory 1440 via a bus 1470. In certain aspects, the computer-readable medium/memory 1440 is configured to store instructions (e.g., computer-executable code) that when executed by the one or more processors 1410, cause the one or more processors 1410 to perform the method 1200 described with respect to FIG. 12, or any aspect related to this method. Note that reference to a processor performing a function of health monitoring device 1400 may include one or more processors 1410 performing that function of health monitoring device 1400.

In the depicted example, computer-readable medium/memory 1440 stores code (e.g., executable instructions), such as code for receiving 1445, code for transmitting 1450, and code for displaying 1455. Processing of the code for receiving 1445, code for transmitting 1450, and code for displaying 1455 may cause the health monitoring device 1400 to perform the method 1200 described with respect to FIG. 12, or any aspect related to this method.

The one or more processors 1410 include circuitry configured to implement (e.g., execute) the code stored in the computer-readable medium/memory 1440, including circuitry for receiving 1415, circuitry for transmitting 1420, and circuitry for displaying 1425. Processing with circuitry for receiving 1415, circuitry for transmitting 1420, and circuitry for displaying 1425 may cause the health monitoring device 1400 to perform the method 1200 described with respect to FIG. 12, or any aspect related to this method.

### Example Clauses

Implementation examples are described in the following numbered clauses:

Clause 1: An analyte sensor system, comprising: an analyte sensor configured to generate analyte data associated with analyte levels of a user of the analyte sensor system; an antenna system comprising plurality of antennas; a transceiver circuit configured to transmit the analyte data to a communications device via one or more antennas of the plurality of antennas of the antenna system; a switching device configured to selectively couple the one or more antennas to the transceiver circuit; and a circuit board configured to operatively connect the analyte sensor with the transceiver circuit.

Clause 2: The analyte sensor system of Clause 1, further comprising: one or more memories; and one or more processors coupled to the one or more memories and the switching device, wherein the one or more processors are configured to cause the analyte sensor system to select the one or more antennas for transmission of the analyte data.

Clause 3: The analyte sensor system of Clause 2, wherein the one or more processors are further configured to cause the analyte sensor system to select, using the switching device, the one or more antennas for transmission of the analyte data based at least in part on channel conditions associated with the plurality of antennas of the antenna system.

Clause 4: The analyte sensor system of any of Clauses 2-3, wherein the one or more processors are further configured to cause the analyte sensor system to receive, from the communications device, feedback indicative of the channel conditions.

Clause 5: The analyte sensor system of Clause 4, wherein the feedback includes a received signal strength indicator (RSSI) associated with each of the antennas of the antenna system.

Clause 6: The analyte sensor system of any of Clauses 2-5, wherein the one or more processors are further configured to cause the analyte sensor system to: receive one or more transmissions from the communications device; and determine angle-of-arrival (AOA) information associated with the one or more transmissions received from the communications device.

Clause 7: The analyte sensor system of Clause 6, further comprising an accelerometer, wherein the one or more processors are further configured to cause the analyte sensor system to determine the AOA information associated with the one or more transmissions received from the communications device based, at least in part, on orientation information from the accelerometer.

Clause 8: The analyte sensor system of any of Clauses 6-7, wherein the one or more processors are further configured to cause the analyte sensor system to select, using the switching device, the one or more antennas for transmission of the analyte data based at least in part on the AOA information associated with the one or more transmissions received from the communications device.

Clause 9: The analyte sensor system of any of Clauses 6-8, wherein: the plurality of antennas of the antenna system comprises at least a first antenna and a second antenna; the first antenna is associated with a first radiation range; and the second antenna is associated with a second radiation range.

Clause 10: The analyte sensor system of Clause 9, wherein the one or more processors are further configured to cause the analyte sensor system to: select the first antenna, using the switching device, when, based on the AOA information, the one or more transmissions are determined to be received in the first radiation rage associated with the first antenna; and select the second antenna, using the switching device, when, based on the AOA information, the one or more transmissions are determined to be received in the second radiation rage associated with the second antenna.

Clause 11: The analyte sensor system of Clause 10, wherein the one or more processors are further configured to cause the analyte sensor system to: use the switching device to couple the second antenna to a ground terminal when the first antenna is selected; and use the switching device to couple the first antenna to the ground terminal when the second antenna is selected.

Clause 12: The analyte sensor system of any of Clauses 1-11, wherein: the plurality of antennas of the antenna system includes a first antenna, a second antenna, and a third antenna; and the third antenna includes the first antenna selectively coupled to at least the second antenna.

Clause 13: The analyte sensor system of Clause 12, wherein: the first antenna is disposed above a first surface of the circuit board; the second antenna is disposed below a second surface of the circuit board; the first antenna is configured to operate as a directing antenna element when the third antenna is used to transmit a signal; and the second antenna is configured to operate as a reflecting antenna element when the first antenna is used to transmit a signal.

Clause 14: The analyte sensor system of any of Clauses 12-13, wherein: the first antenna comprises an inverted-L antenna disposed along a first segment of an edge of the circuit board; and the second antenna comprises a planar inverted-F antenna disposed along a second segment of the edge of the circuit board.

Clause 15: The analyte sensor system of any of Clauses 12-13, wherein: the first antenna comprises a trace antenna; and the second antenna comprises a stamp antenna.

Clause 16: The analyte sensor system of any of Clauses 12-13, wherein: the first antenna comprises a slot antenna; and the second antenna comprises a spiral antenna.

Clause 17: The analyte sensor system of any of Clauses 1-13, wherein the plurality of antennas of the antenna system includes at least one of a patch antenna, a slot antenna, a trace antenna, a spiral antenna, a stamp antenna, an inverted-F antenna, or an inverted-L antenna.

Clause 18: The analyte sensor system of any of Clauses 1-17, wherein the plurality of antennas of the antenna system includes at least one quarter-wave antenna configured to transmit a signal at an operating frequency of 2.4 GHz.

Clause 19: An antenna system for communicating analyte data, comprising: a plurality of antennas; and a switching device configured to selectively couple one or more antennas of the plurality of antennas to a transceiver circuit of an analyte sensor system, wherein, when selectively coupled to the transceiver circuit of the analyte sensor system, the one or more antennas are configured to: receive, from an analyte sensor of the analyte sensor system via the transceiver circuit and a circuit board, analyte data associated with analyte levels of a user of the analyte sensor system; and transmit the analyte data to a communications device for display to the user.

Clause 20: The antenna system of Clause 19, wherein the switching device is configured to select the one or more antennas for transmission of the analyte data based at least in part on channel conditions associated with the plurality of antennas of the antenna system.

Clause 21: The antenna system of Clause 20, wherein at least one of the antennas of the plurality of antennas is configured to receive feedback, from the communications device, indicative of the channel conditions.

Clause 22: The antenna system of Clause 21, wherein the feedback includes a received signal strength indicator (RSSI) associated with each of the antennas of the antenna system.

Clause 23: The antenna system of any of Clauses 19-22, wherein: wherein at least one of the antennas of the plurality of antennas is configured to receive one or more transmissions from the communications device; and the switching device is configured to select the one or more antennas for transmission of the analyte data based at least in part on the angle of arrival (AOA) information associated with the one or more transmissions received from the communications device.

Clause 24: The antenna system of Clause 23, wherein: the plurality of antennas comprises at least a first antenna and a second antenna; the first antenna is associated with a first radiation range; and the second antenna is associated with a second radiation range.

Clause 25: The antenna system of Clause 24, wherein the switching device is configured to: select the first antenna when, based on the AOA information, the one or more transmissions are received in the first radiation rage associated with the first antenna; and select the second antenna when, based on the AOA information, the one or more transmissions are in the second radiation rage associated with the second antenna.

Clause 26: The antenna system of Clause 25, wherein the switching device is configured to: couple the second antenna to a ground terminal when the first antenna is selected; and couple the first antenna to the ground terminal when the second antenna is selected.

Clause 27: The antenna system of any of Clauses 19-26, wherein: the plurality of antennas includes a first antenna, a second antenna, and a third antenna; and the third antenna includes the first antenna selectively coupled to at least the second antenna.

Clause 28: The antenna system of any of Clauses 19-27, wherein the plurality of antennas includes at least one quarter-wave antenna configured to transmit a signal at an operating frequency of 2.4 GHz.

Clause 29: An analyte monitoring system, comprising: a communications device; and an analyte sensor system, comprising: an analyte sensor configured to generate analyte data associated with analyte levels of a user of the analyte sensor system; a first antenna system comprising plurality of antennas; a transceiver circuit configured to transmit the analyte data to a communications device via one or more antennas of the plurality of antennas of the antenna system; a switching device configured to selectively couple the one or more antennas to the transceiver circuit; and a circuit board configured to operatively connect the analyte sensor with the transceiver circuit, wherein: the communications device comprises a second antenna system configured to receive the analyte data from the first antenna system of the analyte sensor system; and the communications device is configured to display the analyte data received from the first antenna system of the analyte sensor system to the user.

Clause 30: The analyte sensor system of Clause 29, further comprising: one or more memories; and one or more processors coupled to the one or more memories and the switching device, wherein the one or more processors are configured to cause the analyte sensor system to select the one or more antennas for transmission of the analyte data.

Clause 31: The analyte sensor system of Clause 30, wherein the one or more processors are further configured to cause the analyte sensor system to select, using the switching device, the one or more antennas for transmission of the analyte data based at least in part on channel conditions associated with the plurality of antennas of the antenna system.

Clause 32: The analyte sensor system of any of Clauses 30-31, wherein the one or more processors are further configured to cause the analyte sensor system to receive, from the communications device, feedback indicative of the channel conditions.

Clause 33: The analyte sensor system of any of Clause 32, wherein the feedback includes a received signal strength indicator (RSSI) associated with each of the antennas of the antenna system.

Clause 34: The analyte sensor system of any of Clauses 30-33, wherein the one or more processors are further configured to cause the analyte sensor system to: receive one or more transmissions from the communications device; and determine angle-of-arrival (AOA) information associated with the one or more transmissions received from the communications device.

Clause 35: The analyte sensor system of Clause 34, further comprising an accelerometer, wherein the one or more processors are further configured to cause the analyte sensor system to determine the AOA information associated with the one or more transmissions received from the communications device using the accelerometer.

Clause 36: The analyte sensor system of any of Clauses 34-35, wherein the one or more processors are further configured to cause the analyte sensor system to select, using the switching device, the one or more antennas for transmission of the analyte data based at least in part on the AOA information associated with the one or more transmissions received from the communications device.

Clause 37: The analyte sensor system of any of Clauses 34-36, wherein: the plurality of antennas of the antenna system comprises at least a first antenna and a second antenna; the first antenna is associated with a first radiation range; and the second antenna is associated with a second radiation range.

Clause 38: The analyte sensor system of Clause 37, wherein the one or more processors are further configured to cause the analyte sensor system to: select the first antenna, using the switching device, when, based on the AOA information, the one or more transmissions are determined to be received in the first radiation rage associated with the first antenna; and select the second antenna, using the switching device, when, based on the AOA information, the one or more transmissions are determined to be received in the second radiation rage associated with the second antenna.

Clause 39: The analyte sensor system of Clause 38, wherein the one or more processors are further configured to cause the analyte sensor system to: use the switching device to couple the second antenna to a ground terminal when the first antenna is selected; and use the switching device to couple the first antenna to the ground terminal when the second antenna is selected.

Clause 40: The analyte sensor system of any of Clauses 29-39, wherein: the plurality of antennas of the antenna system includes a first antenna, a second antenna, and a third antenna; and the third antenna includes the first antenna selectively coupled to at least the second antenna.

Clause 41: A method for communication by an analyte sensor system, comprising: generating analyte data associated with analyte levels of a user of the analyte sensor system; selecting, using a switching device of the analyte sensor system, a first antenna of a plurality of antennas of an antenna system of the analyte sensor system for transmission of the analyte data; transmitting the analyte data to a communications device using the first antenna; selecting, using the switching device, at least a second antenna of the plurality of antennas of the antenna system; and transmitting the analyte data to the communications device using at least the second antenna.

Clause 42: The method of Clause 41, wherein selecting, using the switching device, the first antenna and at least the second antenna is based at least in part on channel conditions associated with the plurality of antennas of the antenna system.

Clause 43: The method of any of Clauses 41-42, further comprising receiving, from the communications device, feedback indicative of the channel conditions.

Clause 44: The method of Clause 43, wherein the feedback includes a received signal strength indicator (RSSI) associated with each of the antennas of the antenna system.

Clause 45: The method of any of Clauses 41-44, further comprising: receiving one or more transmissions from the communications device; and determining angle-of-arrival (AOA) information associated with the one or more transmissions received from the communications device.

Clause 46: The method of Clause 45, wherein determining the AOA information associated with the one or more transmissions received from the communications device is based, at least in part, on orientation information from an accelerometer of the analyte sensor system.

Clause 47: The method of any of Clauses 45-46, wherein selecting, using the switching device, the first antenna and at least the second antenna is based at least in part on the AOA information associated with the one or more transmissions received from the communications device.

Clause 48: The method of any of Clauses 45-47, wherein: the first antenna is associated with a first radiation range; and the second antenna is associated with a second radiation range.

Clause 49: The method of Clause 48, wherein: selecting the first antenna, using the switching device, comprises selecting the first antenna when, based on the AOA information, the one or more transmissions are determined to be received in the first radiation rage associated with the first antenna; and selecting the second antenna, using the switching device, comprises selecting the second antenna when, based on the AOA information, the one or more transmissions are determined to be received in the second radiation rage associated with the second antenna.

Clause 50: The method of Clause 49, further comprising: using the switching device to couple the second antenna to a ground terminal when the first antenna is selected; and using the switching device to couple the first antenna to the ground terminal when the second antenna is selected.

Clause 51: The method of any of Clauses 41-50, wherein: the plurality of antennas of the antenna system includes a third antenna; and the third antenna includes the first antenna selectively coupled to at least the second antenna.

Clause 52: The method of Clause 51, wherein: the first antenna is disposed above a first surface of a circuit board of the analyte sensor system; the second antenna is disposed below a second surface of the circuit board; the first antenna is configured to operate as a directing antenna element when the third antenna is used to transmit a signal; and the second antenna is configured to operate as a reflecting antenna element when the first antenna is used to transmit a signal.

Clause 53: The method of any of Clauses 51-52, wherein: the first antenna comprises an inverted-L antenna disposed along a first segment of an edge of the circuit board; and the second antenna comprises a planar inverted-F antenna disposed along a second segment of the edge of the circuit board.

Clause 54: The method of any of Clauses 51-52, wherein: the first antenna comprises a trace antenna; and the second antenna comprises a stamp antenna.

Clause 55: The method of any of Clauses 51-52, wherein: the first antenna comprises a slot antenna; and the second antenna comprises a spiral antenna.

Clause 56: The method of Clauses 41-52, wherein the plurality of antennas of the antenna system includes at least one of a patch antenna, a slot antenna, a trace antenna, a spiral antenna, a stamp antenna, an inverted-F antenna, or an inverted-L antenna.

Clause 57: The method of any of Clauses 41-56, wherein the plurality of antennas of the antenna system includes at least one quarter-wave antenna configured to transmit a signal at an operating frequency of 2.4 GHz.

Clause 58: A method for communication between a communications device and an analyte sensor system in an analyte monitoring system, comprising: receiving, by the communications device, analyte data from the analyte sensor system in a first position; failing to receive, by the communications device, the analyte data from the analyte sensor system in a second position different from the first position; switching, by the analyte sensor system, an antenna for transmission of the analyte data; and receiving, by the communications device, the analyte data from the analyte sensor system in the second position based on switching the antenna.

Clause 59: The method of Clause 58, wherein switching, by the analyte sensor system, the antenna for transmission of the analyte data comprises switching, using a switching device of the analyte sensor system, from a first antenna of a plurality of antennas of an antenna system of the analyte sensor system to a second antenna of the plurality of antennas of the antenna system.

Clause 60: The method of Clause 59, wherein switching from the first antenna to the second antenna is based at least in part on channel conditions associated with the plurality of antennas of the antenna system.

Clause 61: The method of any of Clauses 58-60, further comprising: transmitting, by the communications device, feedback indicative of the channel conditions; and receiving, by the analyte sensor system, the feedback indicative of the channel conditions.

Clause 62: The method of Clause 61, wherein the feedback includes a received signal strength indicator (RSSI) associated with each of the antennas of the antenna system.

Clause 63: The method of any of Clauses 58-62, further comprising: transmitting, by the communications device, one or more transmissions; receiving, by the analyte sensor system, the one or more transmissions from the communications device; and determining, by the analyte sensor system, angle-of-arrival (AOA) information associated with the one or more transmissions received from the communications device.

Clause 64: The method of Clause 63, wherein determining the AOA information associated with the one or more transmissions received from the communications device is based, at least in part, on orientation information from an accelerometer of the analyte sensor system.

Clause 65: The method of any of Clauses 63-64, wherein switching, by the analyte sensor system, from the first antenna to the second antenna is based at least in part on the AOA information associated with the one or more transmissions received from the communications device.

Clause 66: The method of any of Clauses 63-65, wherein: the first antenna is associated with a first radiation range; and the second antenna is associated with a second radiation range.

Clause 67: The method of Clause 66, wherein switching, by the analyte sensor system, from the first antenna to the second antenna comprises switching to the second antenna when, based on the AOA information, the one or more transmissions are determined, by the analyte sensor system, to be received in the second radiation rage associated with the second antenna.

Clause 68: The method of Clause 67, further comprising switching, by the analyte sensor system, from the second antenna to the first antenna when, based on the AOA information, the one or more transmissions are determined, by the analyte sensor system, to be received in the first radiation rage associated with the first antenna.

Clause 69: The method of Clause 68, further comprising: using, by the analyte sensor system, the switching device to couple the second antenna to a ground terminal when the first antenna is selected; and using, by the analyte sensor system, the switching device to couple the first antenna to the ground terminal when the second antenna is selected.

Clause 70: The method of any of Clauses 58-69, wherein: the plurality of antennas of the antenna system includes a third antenna; and the third antenna includes the first antenna selectively coupled to at least the second antenna.

Clause 71: The method of Clause 70, wherein: the first antenna is disposed above a first surface of a circuit board of the analyte sensor system; the second antenna is disposed below a second surface of the circuit board; the first antenna is configured to operate as a directing antenna element when the third antenna is used to transmit a signal; and the second antenna is configured to operate as a reflecting antenna element when the first antenna is used to transmit a signal.

Clause 72: The method of any of Clauses 70-71, wherein: the first antenna comprises an inverted-L antenna disposed along a first segment of an edge of the circuit board; and the second antenna comprises a planar inverted-F antenna disposed along a second segment of the edge of the circuit board.

Clause 73: The method of any of Clauses 70-71, wherein: the first antenna comprises a trace antenna; and the second antenna comprises a stamp antenna.

Clause 74: The method of any of Clauses 70-71, wherein: the first antenna comprises a slot antenna; and the second antenna comprises a spiral antenna.

Clause 75: The method of any of Clauses 58-71, wherein the plurality of antennas of the antenna system includes at least one of a patch antenna, a slot antenna, a trace antenna, a spiral antenna, a stamp antenna, an inverted-F antenna, or an inverted-L antenna.

Clause 76: The method of any of Clauses 58-75, wherein the plurality of antennas of the antenna system includes at least one quarter-wave antenna configured to transmit a signal at an operating frequency of 2.4 GHz.

Clause 77: The method of any of Clauses 58-76, further comprising displaying, by the communications device, the analyte data to a user.

Clause 78: An apparatus, comprising: a memory comprising executable instructions; and one or more processors configured to execute the executable instructions and cause the apparatus to perform a method in accordance with any one of Clauses 41-77.

Clause 79: An apparatus, comprising means for performing a method in accordance with any one of Clauses 41-77.

Clause 80: A non-transitory computer-readable medium comprising executable instructions that, when executed by one or more processors of an apparatus, cause the apparatus to perform a method in accordance with any one of Clauses 41-77.

Clause 81: A computer program product embodied on a computer-readable storage medium comprising code for performing a method in accordance with any one of Clauses 41-77.

### ADDITIONAL CONSIDERATIONS

In this document, the terms “computer program medium” and “computer usable medium” and “computer readable medium”, as well as variations thereof, are used to generally refer to transitory or non-transitory media. These and other various forms of computer program media or computer usable/readable media may be involved in carrying one or more sequences of one or more instructions to a processing device for execution. Such instructions embodied on the medium, may generally be referred to as “computer program code” or a “computer program product” or “instructions” (which may be grouped in the form of computer programs or other groupings). When executed, such instructions may enable a computing module, such as the analyte sensor system 208 and/or analyte sensor system 700, circuitry related thereto, and/or a processor thereof or connected thereto to perform features or functions of the present disclosure as discussed herein (for example, in connection with methods described above and/or in the claims), including for example when the same is/are incorporated into a system, apparatus, device and/or the like.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense. It will be appreciated that, for clarity purposes, the above description has described embodiments with reference to different functional units. However, it will be apparent that any suitable distribution of functionality between different functional units may be used without detracting from the invention. For example, functionality illustrated to be performed by separate computing devices may be performed by the same computing device. Likewise, functionality illustrated to be performed by a single computing device may be distributed amongst several computing devices. Hence, references to specific functional units are only to be seen as references to suitable means for providing the described functionality, rather than indicative of a strict logical or physical structure or organization.

Although described above in terms of various example embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof, the terms “a” or “an” should be read as meaning “at least one,” “one or more” or the like; the term “set” should be read to include one or more objects of the type included in the set; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Similarly, the plural may in some cases be recognized as applicable to the singular and vice versa. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic, circuitry, or other components, may be combined in a single package or separately maintained and may further be distributed in multiple groupings or packages or across multiple locations.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration. Moreover, the operations and sub-operations of various methods described herein are not necessarily limited to the order described or shown in the figures, and one of skill in the art will appreciate, upon studying the present disclosure, variations of the order of the operations described herein that are within the spirit and scope of the disclosure. It will be understood that each block of the flowchart illustrations, and combinations of blocks in the flowchart illustrations, can be implemented by execution of computer program instructions. These computer program instructions may be loaded onto a computer or other programmable data processing apparatus (such as a controller, microcontroller, microprocessor or the like) in a sensor electronics system to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks presented herein.

It should be appreciated that all methods and processes disclosed herein may be used in any glucose or other analyte monitoring system, continuous or intermittent. It should further be appreciated that the implementation and/or execution of all methods and processes may be performed by any suitable devices or systems, whether local or remote. Further, any combination of devices or systems may be used to implement the present methods and processes.

In addition, the operations and sub-operations of methods described herein may be carried out or implemented, in some cases, by one or more of the components, elements, devices, modules, circuitry, processors, etc. of systems, apparatuses, devices, environments, and/or computing modules described herein and referenced in various of figures of the present disclosure, as well as one or more sub-components, elements, devices, modules, processors, circuitry, and the like depicted therein and/or described with respect thereto. In such instances, the description of the methods or aspects thereof may refer to a corresponding component, element, etc., but regardless of whether an explicit reference is made, one of skill in the art will recognize upon studying the present disclosure when the corresponding component, element, etc. may be used. Further, it will be appreciated that such references do not necessarily limit the described methods to the particular component, element, etc. referred to. Thus, it will be appreciated by one of skill in the art that aspects and features described above in connection with (sub-) components, elements, devices, modules, and circuitry, etc., including variations thereof, may be applied to the various operations described in connection with methods described herein, and vice versa, without departing from the scope of the present disclosure.

1. An analyte sensor system, comprising:
an analyte sensor configured to generate analyte data associated with analyte levels of a user of the analyte sensor system;
an antenna system comprising plurality of antennas;
a transceiver circuit configured to transmit the analyte data to a communications device via one or more antennas of the plurality of antennas of the antenna system;
a switching device configured to selectively couple the one or more antennas to the transceiver circuit; and
a circuit board configured to operatively connect the analyte sensor with the transceiver circuit.

2. The analyte sensor system of claim 1, further comprising:
one or more memories; and
one or more processors coupled to the one or more memories and the switching device, wherein the one or more processors are configured to cause the analyte sensor system to select the one or more antennas for transmission of the analyte data.

3. The analyte sensor system of claim 2, wherein the one or more processors are further configured to cause the analyte sensor system to select, using the switching device, the one or more antennas for transmission of the analyte data based at least in part on channel conditions associated with the plurality of antennas of the antenna system.

4. The analyte sensor system of claim 2, wherein the one or more processors are further configured to cause the analyte sensor system to receive, from the communications device, feedback indicative of the channel conditions.

5. The analyte sensor system of claim 4, wherein the feedback includes a received signal strength indicator (RSSI) associated with each of the antennas of the antenna system.

6. The analyte sensor system of claim 2, wherein the one or more processors are further configured to cause the analyte sensor system to:
receive one or more transmissions from the communications device; and
determine angle-of-arrival (AOA) information associated with the one or more transmissions received from the communications device.

7. The analyte sensor system of claim 6, further comprising an accelerometer, wherein the one or more processors are further configured to cause the analyte sensor system to determine the AOA information associated with the one or more transmissions received from the communications device based, at least in part, on orientation information from the accelerometer.

8. The analyte sensor system of claim 6, wherein the one or more processors are further configured to cause the analyte sensor system to select, using the switching device, the one or more antennas for transmission of the analyte data based at least in part on the AOA information associated with the one or more transmissions received from the communications device.

9. The analyte sensor system of claim 6, wherein:
the plurality of antennas of the antenna system comprises at least a first antenna and a second antenna;
the first antenna is associated with a first radiation range; and
the second antenna is associated with a second radiation range.

10. The analyte sensor system of claim 9, wherein the one or more processors are further configured to cause the analyte sensor system to:
select the first antenna, using the switching device, when, based on the AOA information, the one or more transmissions are determined to be received in the first radiation rage associated with the first antenna; and
select the second antenna, using the switching device, when, based on the AOA information, the one or more transmissions are determined to be received in the second radiation rage associated with the second antenna.

11. The analyte sensor system of claim 10, wherein the one or more processors are further configured to cause the analyte sensor system to:
use the switching device to couple the second antenna to a ground terminal when the first antenna is selected; and
use the switching device to couple the first antenna to the ground terminal when the second antenna is selected.

12. The analyte sensor system of claim 1, wherein:
the plurality of antennas of the antenna system includes a first antenna, a second antenna, and a third antenna; and
the third antenna includes the first antenna selectively coupled to at least the second antenna.

13. The analyte sensor system of claim 12, wherein:
the first antenna is disposed above a first surface of the circuit board;
the second antenna is disposed below a second surface of the circuit board;
the first antenna is configured to operate as a directing antenna element when the third antenna is used to transmit a signal; and
the second antenna is configured to operate as a reflecting antenna element when the first antenna is used to transmit a signal.

14. The analyte sensor system of claim 12, wherein:
the first antenna comprises an inverted-L antenna disposed along a first segment of an edge of the circuit board; and
the second antenna comprises a planar inverted-F antenna disposed along a second segment of the edge of the circuit board.

15. The analyte sensor system of claim 12, wherein:
the first antenna comprises a trace antenna; and
the second antenna comprises a stamp antenna.

16. The analyte sensor system of claim 12, wherein:
the first antenna comprises a slot antenna; and
the second antenna comprises a spiral antenna.

17. The analyte sensor system of claim 1, wherein the plurality of antennas of the antenna system includes at least one of a patch antenna, a slot antenna, a trace antenna, a spiral antenna, a stamp antenna, an inverted-F antenna, or an inverted-L antenna.

18. The analyte sensor system of claim 1, wherein the plurality of antennas of the antenna system includes at least one quarter-wave antenna configured to transmit a signal at an operating frequency of 2.4 GHz.

19. An antenna system for communicating analyte data, comprising:
a plurality of antennas; and
a switching device configured to selectively couple one or more antennas of the plurality of antennas to a transceiver circuit of an analyte sensor system, wherein, when selectively coupled to the transceiver circuit of the analyte sensor system, the one or more antennas are configured to:
receive, from an analyte sensor of the analyte sensor system via the transceiver circuit and a circuit board, analyte data associated with analyte levels of a user of the analyte sensor system; and
transmit the analyte data to a communications device for display to the user.

20-28. (canceled)

29. An analyte monitoring system, comprising:
a communications device; and
an analyte sensor system, comprising:
an analyte sensor configured to generate analyte data associated with analyte levels of a user of the analyte sensor system;
a first antenna system comprising plurality of antennas;
a transceiver circuit configured to transmit the analyte data to a communications device via one or more antennas of the plurality of antennas of the antenna system;
a switching device configured to selectively couple the one or more antennas to the transceiver circuit; and
a circuit board configured to operatively connect the analyte sensor with the transceiver circuit, wherein:
the communications device comprises a second antenna system configured to receive the analyte data from the first antenna system of the analyte sensor system; and
the communications device is configured to display the analyte data received from the first antenna system of the analyte sensor system to the user.

30-77. (canceled)
